(19) United States
(12) Patent Application Publication
(10) Pub. No.: US 2026/0207794 A1
(43) Pub. Date: Jul. 23, 2026

(54) NOVEL COMPOUNDS FOR DIAGNOSIS

(71) Applicant: AC IMMUNE SA, Lausanne (CH)

(72) Inventor: Jérôme Molette, Lausanne (CH)

(21) Appl. No.: 19/139,657

(22) PCT Filed: Dec. 15, 2023
(86) PCT No.: PCT/EP2023/086191

§ 371 Date: Jun. 16, 2025

(30) Foreign Application Priority Data
Dec. 16, 2022 (EP) 22214289.5

(51) Int. Cl. A61K 51/04; A61B 6/03; A61B 6/50; A61P 25/16; C07B 59/00; C07D 487/04

(52) U.S. Cl. CPC A61K 51/0455; A61B 6/037; A61B 6/501; A61P 25/16; C07B 59/002; C07D 487/04

(57) ABSTRACT

The present invention relates to novel compounds of formula (I), or a detectably labelled compound, stereoisomer, racemic mixture, pharmaceutically acceptable salt, hydrate, or solvate thereof, that can be employed in the imaging of alpha-synuclein aggregates and determining an amount thereof. Furthermore, the compounds can be used for diagnosing a disease, disorder or abnormality associated with an alpha-synuclein aggregates (such as Parkinson's disease or such as multiple system atrophy (MSA)) determining a predisposition to such a disease, disorder or abnormality, prognosing such a disease, disorder or abnormality, monitoring the evolution of the disease in a patient suffering from such a disease, disorder or abnormality, monitoring the progression of such a disease, disorder or abnormality and predicting responsiveness of a patient suffering from such a disease, disorder or abnormality to a treatment thereof.

### FIELD OF THE INVENTION

The present invention relates to novel compounds of formula (I), or a detectably labelled compound, stereoisomer, racemic mixture, pharmaceutically acceptable salt, hydrate, or solvate thereof, that can be employed in the imaging of alpha-synuclein aggregates and determining an amount thereof. Furthermore, the compounds can be used for diagnosing a disease, disorder or abnormality associated with alpha-synuclein (α-synuclein, A-synuclein, aSynuclein, A-syn, α-syn, aSyn, a-syn) aggregates, such as Parkinson's disease or multiple system atrophy (MSA), determining a predisposition to such a disease, disorder or abnormality, prognosing such a disease, disorder or abnormality, monitoring the evolution of the disease in a patient suffering from such a disease, disorder or abnormality, monitoring the progression of such a disease, disorder or abnormality and predicting responsiveness of a patient suffering from such a disease, disorder or abnormality to a treatment thereof. The present invention also relates to processes for the preparation of the compounds and their precursors, diagnostic compositions comprising the compounds, methods of using the compounds, kits comprising the compounds and their uses thereof.

### BACKGROUND OF THE INVENTION

Many diseases of aging are based on or associated with extracellular or intracellular deposits of amyloid or amyloid-like proteins that contribute to the pathogenesis as well as to the progression of the disease. The best characterized amyloid protein that forms extracellular aggregates is amyloid beta (Abeta or Aβ).

Amyloid-like proteins that form mainly intracellular aggregates include, but are not limited to, Tau, alpha-synuclein, and huntingtin (HTT). Diseases involving alpha-synuclein aggregates are generally listed as synucleinopathies (or alpha-synucleinopathies) and these include, but are not limited to, Parkinson's disease (PD) or multiple system atrophy (MSA). Synucleinopathies with primarily neuronal aggregates include, but are not limited to, Parkinson's disease (sporadic, familial with SNCA (the gene encoding for the alpha-synuclein protein) mutations or SNCA gene duplication or triplication, familial with mutations in other genes than SNCA, pure autonomic failure and Lewy body dysphagia), SNCA duplication carrier, Lewy Body dementia (LBD), dementia with Lewy bodies (DLB) (“pure” Lewy body dementia), Parkinson's disease dementia (PDD), diffuse Lewy body disease (DLBD), Alzheimer's disease, sporadic Alzheimer's disease, familial Alzheimer's disease with APP mutations, familial Alzheimer's disease with PS-1, PS-2 or other mutations, familial British dementia, Lewy body variant of Alzheimer's disease and normal aging in Down syndrome. Synucleinopathies with neuronal and glial aggregates of alpha-synuclein include, but are not limited to, multiple system atrophy (MSA) (Shy-Drager syndrome, striatonigral degeneration and olivopontocerebellar atrophy). Other diseases that may have alpha-synuclein-immunoreactive lesions are, but are not limited to, traumatic brain injury, chronic traumatic encephalopathy, dementia pugilistica, tauopathies (Pick's disease, frontotemporal dementia, progressive supranuclear palsy, corticobasal degeneration and Niemann-Pick type C1 disease, frontotemporal dementia with Parkinsonism linked to chromosome 17), motor neuron disease, Huntington's disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Creutzfeldt-Jakob disease, ataxia telangiectatica, Meige's syndrome, subacute sclerosing panencephalitis, Gerstmann-Straussler-Scheinker disease, inclusion-body myositis, Gaucher disease, Krabbe disease as well as other lysosomal storage disorders (including Kufor-Rakeb syndrome and Sanfilippo syndrome) and rapid eye movement (REM) sleep behavior disorder (Jellinger, Mov. Disord. 2003, 18 Suppl. 6, S2-12; Galvin et al. JAMA Neurology 2001, 58 (2), 186-190; Kovari et al., Acta Neuropathol. 2007, 114(3), 295-8; Saito et al., J. Neuropathol. Exp. Neurol. 2004, 63(4), 323-328; McKee et al., Brain, 2013, 136(Pt 1), 43-64; Puschmann et al., Parkinsonism Relat. Disord. 2012, 18S1, S24-S27; Usenovic et al., J. Neurosci. 2012, 32(12), 4240-4246; Winder-Rhodes et al., Mov. Disord. 2012, 27(2), 312-315; Ferman et al., J. Int. Neuropsychol. Soc. 2002, 8(7), 907-914; Smith et al., J. Pathol. 2014; 232:509-521, Lippa et al., Ann Neurol. 1999 March; 45(3):353-7; Schmitz et al., Mol. Neurobiol. 2018 Aug. 22; Charles et al., Neurosci. Lett. 2000 Jul. 28; 289(1):29-32; Wilhelmsen et al., Arch Neurol. 2004 March; 61(3):398-406; Yamaguchi et al., J. Neuropathol. Exp. Neurol. 2004, 80^{th} annual meeting, vol. 63; Askanas et al., J. Neuropathol. Exp. Neurol. 2000 July; 59(7):592-8).

Alpha-synuclein is a 140 amino acid natively unfolded protein (Iwai et al., Biochemistry 1995, 34(32), 10139-10145). The sequence of alpha-synuclein can be divided into three main domains: 1) the N-terminal region comprising of residues 1-60, which contains the 11-mer amphipatic imperfect repeat residues with highly conserved hexamer (KTKEGV). This region has been implicated in regulating alpha-synuclein binding to membranes and its internalization; 2) the hydrophobic Non Amyloid beta Component (NAC) domain spanning residues 61-95; which is essential for alpha-synuclein fibrillization; and 3) the C-terminal region spanning residues 96-140 which is highly acidic and proline-rich and has no distinct structural propensity. Alpha-synuclein has been shown to undergo several posttranslational modifications, including truncations, phosphorylation, ubiquitination, oxidation and/or transglutaminase covalent cross linking (Fujiwara et al., Nat. Cell. Biol. 2002, 4(2); 160-164; Hasegawa et al., J. Biol. Chem. 2002, 277(50), 49071-49076; Li et al., Proc. Natl. Acad. Sci. USA 2005, 102(6), 2162-2167; Oueslati et al., Prog. Brain Res. 2010, 183, 115-145; Schmid et al., J. Biol. Chem. 2009, 284(19), 13128-13142). Interestingly, the majority of these modifications involve residues within the C-terminal region.

Several phosphorylation sites have been detected in the carboxyl-terminal region on Tyr-125, -133, and -136, and on Ser-129 (Negro et al., FASEB J. 2002, 16(2), 210-212). Tyr-125 residues can be phosphorylated by two Src family protein tyrosine kinases, c-Src and Fyn (Ellis et al., J. Biol. Chem. 2001, 276(6), 3879-3884; Nakamura et al., Biochem. Biophys. Res. Commun. 2001, 280(4), 1085-1092). Phosphorylation by Src family kinases does not suppress or enhance the tendency of alpha-synuclein to polymerize. Alpha-synuclein has proved to be an outstanding substrate for protein tyrosine kinase p72^{syk} (Syk) in vitro; once it is extensively Tyr-phosphorylated by Syk or tyrosine kinases with similar specificity, it loses the ability to form oligomers, suggesting a putative anti-neurodegenerative role for these tyrosine kinases (Negro et al., FASEB J. 2002, 16(2), 210-212). Alpha-synuclein can be Ser-phosphorylated by protein kinases CKI and CKII (Okochi et al., J. Biol. Chem. 2000, 275(1), 390-397). The residue Ser-129 is also phosphorylated by G-protein-coupled receptor protein kinases (Pronin et al., J. Biol. Chem. 2000, 275(34), 26515-26522). Extensive and selective phosphorylation of alpha-synuclein at Ser-129 is evident in synucleinopathy lesions, including Lewy bodies (Fujiwara et al., Nat. Cell. Biol. 2002, 4(2); 160-164). Other post-translational modifications in the carboxyl-terminal, including glycosylation on Ser-129 (McLean et al., Neurosci. Lett. 2002, 323(3), 219-223) and nitration on Tyr-125, -133, and -136 (Takahashi et al., Brain Res. 2002, 938(1-2), 73-80), may affect aggregation of alpha-synuclein. Truncation of the carboxyl-terminal region by proteolysis has been reported to play a role in alpha-synuclein fibrillogenesis in various neurodegenerative diseases (Rochet et al., Biochemistry 2000, 39(35), 10619-10626). Full-length as well as partially truncated and insoluble aggregates of alpha-synuclein have been detected in highly purified Lewy bodies (Crowther et al., FEBS Lett. 1998, 436(3), 309-312).

Abnormal protein aggregation appears to be a common feature in aging brain and in several neurodegenerative diseases (Trojanowski et al., 1998, Cell Death Differ. 1998, 5(10), 832-837, Koo et al., Proc. Natl. Acad. Sci. 1999, 96(18), 9989-9990, Hu et al., Chin. Sci. Bull. 2001, 46, 1-3); although a clear role in the disease process remains to be defined. In in vitro models, alpha-synuclein (or some of its truncated forms) readily assembles into filaments resembling those isolated from the brain of patients with Lewy Body (LB) dementia and familiar PD (Crowther et al., FEBS Lett. 1998, 436(3), 309-312). Alpha-synuclein and its mutated forms (A53T and A30P) have a random coil conformation and do not form significant secondary structures in aqueous solution at low concentrations; however, at higher concentrations they are prone to self-aggregate, producing amyloid fibrils (Wood et al., J. Biol. Chem. 1999, 274(28), 19509-19512). Several differences in the aggregation behavior of the PD-linked mutants and the wild-type protein have been documented. Monomeric alpha-synuclein aggregates in vitro form stable fibrils via a metastable oligomeric (i.e., protofibril) state (Volles et al., Biochemistry 2002, 41(14), 4595-4602).

Parkinson's disease (PD) is the most common neurodegenerative motor disorder. PD is mainly an idiopathic disease, although in at least 5% of the PD patients the pathology is linked to mutations in one or several specific genes. Several point mutations have been described in the alpha-synuclein gene (A30P, E46K, H50Q, G51D, A53T) which cause familial PD with autosomal dominant inheritance. Furthermore, duplications and triplications of the alpha-synuclein gene have been described in patients that developed PD, underlining the role of alpha-synuclein in PD pathogenesis (Lesage et al., Hum. Mol. Genet., 2009, 18, R48-59). The pathogenesis of PD remains elusive. However, growing evidence suggests a role for the pathogenic folding of the alpha-synuclein protein that leads to the formation of amyloid-like fibrils. Indeed, the hallmarks of PD are the presence of intracellular alpha-synuclein aggregate structures called Lewy Bodies and neurites mainly in the nigral neurons, as well as the death of dopaminergic neurons in the substantia nigra and elsewhere. Alpha-synuclein is a natively unfolded presynaptic protein that can misfold and aggregate into larger oligomeric and fibrillar forms which are linked to the pathogenesis of PD. Recent studies have implicated small soluble oligomeric and protofibrillar forms of alpha-synuclein as the most neurotoxic species (Lashuel et al., J. Mol. Biol., 2002, 322, 1089-102). However, the precise role of alpha-synuclein in the neuronal cell toxicity remains to be clarified (review: Cookson, Annu. Rev. Biochem., 2005, 74, 29-52).

Besides Parkinson's disease, the accumulation of aggregated alpha-synuclein into Lewy bodies is a characteristic of all Lewy body diseases, including Parkinson's disease with dementia (PDD), and dementia with Lewy bodies (DLB) (Capouch et al., Neurol. Ther. 2018, 7, 249-263). In DLB, Lewy Bodies are diffusely distributed throughout the cortices of the brain and in addition to Lewy bodies and neurites, more threads and dot-like structures (Lewy dots) were found to be immunopositive for alpha-synuclein phosphorylated at Ser-129 (Outeiro et al., Mol. Neurodegener. 2019, 14, 5).

Alpha-synuclein aggregates are also found in multiple system atrophy (MSA). MSA is a rare and sporadic neurodegenerative disorder that manifests with rapidly progressive autonomic and motor dysfunction, as well as variable cognitive decline. Such disorders include Shy-Drager syndrome, striatonigral degeneration and olivopontocerebellar atrophy. The disease can be clinically sub-classified in parkinsonian (MSA-P) or cerebellar (MSA-C) variant, depending on the predominant motor phenotype (Fanciulli et al., N. Engl. J. Med. 2015; 372, 249-63). It is characterized by the aggregation of alpha-synuclein in the cytoplasm of oligodendrocytes, forming glial cytoplasmic inclusions (GCIs). GCIs, consisting primarily of fibrillary forms of alpha-synuclein, are the neuropathological hallmark of MSA and are found throughout the neocortex, hippocampus, brainstem, spinal cord and dorsal root ganglia (Galvin et al., Arch Neurol. 2001, 58, 186-90). GCIs are considered a central player in the pathogenesis of MSA. A correlation between the GCI load and the degree of neuronal loss has been reported in both the striatonigral and the olivopontocerebellar regions (Stefanova et al., Neuropathol. Appl. Neurobiol. 2016, 42, 20-32).

Furthermore, a causative link between GCIs and the induction of neuronal loss has been shown in transgenic mice overexpressing human alpha-synuclein in oligodendrocytes under various oligodendroglia-specific promoters. A key event in the pathophysiological cascade is considered to be the permissive templating (‘prion-like’ propagation) of misfolded alpha-synuclein.

The diagnosis of Parkinson's disease is largely clinical and depends on the presence of a specific set of symptoms and signs (the initial core feature being bradykinesia, rigidity, rest tremor and postural instability), the absence of atypical features, a slowly progressive course, and the response to a symptomatic drug therapy, mainly limited to a dopamine replacement therapy. The accurate diagnosis requires sophisticated clinical skills and is open to a degree of subjectivity and error, as several other degenerative and non-degenerative diseases can mimic PD symptoms (multiple system atrophy (MSA), progressive supranuclear palsy (PSP), Alzheimer's disease (AD), essential tremor, dystonic tremor), (Guideline No. 113: Diagnosis and pharmacological management of Parkinson's disease, January 2010. SIGN). The final confirmation of the pathology can only be made by post-mortem neuropathological analysis.

Computed tomography (CT) and conventional magnetic resonance imaging (MRI) brain scans of people with Parkinson's disease (PD) usually appear normal. These techniques are nevertheless useful to rule out other diseases that can be secondary causes of parkinsonism, such as basal ganglia tumors, vascular pathology and hydrocephalus. A specific technique of MRI, diffusion MRI, has been reported to be useful at discriminating between typical and atypical parkinsonism, although its exact diagnostic value is still under investigation. Dopaminergic function in the basal ganglia can be measured with different PET and SPECT radiotracers. Examples are ioflupane (¹²³I) (trade name DaTSCAN) and iometopane (Dopascan) for SPECT or fluorodeoxyglucose (¹⁸F) (¹⁸F-FDG) and dihydrotetrabenazine (¹¹C) (¹¹C-DTBZ) for PET. A pattern of reduced dopaminergic activity in the basal ganglia can aid in diagnosing PD, particularly in the symptomatic stage (Brooks, J. Nucl. Med., 2010, 51, 596-609; Redmond, Neuroscientist, 2002, 8, 457-88; Wood, Nat. Rev. Neurol., 2014, 10, 305).

Strategies are being developed to apply recent advances in understanding the potential causes of Parkinson's disease to the development of biochemical biomarkers (Schapira Curr. Opin. Neurol. 2013; 26(4):395-400). Such biomarkers that have been investigated in different body fluids (cerebrospinal fluid (CSF), plasma, saliva) include alpha-synuclein levels but also DJ-1, Tau and Abeta, as well as neurofilaments proteins, interleukins, osteopontin and hypocrontin (Schapira Curr. Opin. Neurol. 2013; 26(4):395-400), but so far none of these biomarkers alone or in combination can be used as a determinant diagnostic test. To our knowledge, no approved alpha-synuclein diagnostic agent is currently on the market despite a crucial need for Parkinson's disease research and drug development (Eberling et al., J Parkinsons Dis. 2013; 3(4):565-7).

The ability to image alpha-synuclein deposition in the brain would be a huge achievement for alpha-synucleopathies research, including Parkinson's disease (PD) and MSA research, diagnosis, and drug development. The accumulation of aggregated alpha-synuclein in the brain is considered a key pathological hallmark of PD and MSA and can start many years before the appearance of the symptoms. Therefore, alpha-synuclein is a priority target for drug development given not only its likely contribution to neurodegeneration but also because it can offer the possibility to treat the disease while still in the asymptomatic or prodromal stages. In vivo imaging of alpha-synuclein pathology could be useful as a biomarker to (i) detect the presence of the disease potentially in early stages, (ii) to evaluate disease progression and (iii) to be used as a pharmacodynamics tool for drug development. The development of an alpha-synuclein PET imaging agent is considered nowadays key for an accurate diagnosis of synucleinopathies as well as to support the clinical development of therapeutics targeting alpha-synuclein, starting from the optimal selection of the trial population (Eberling, Dave and Frasier, J. Parkinson's Disease, 3, 565-567 (2013)).

Only recently, the first non-invasive images of pathological alpha-synuclein (a-syn) in human brain were reported and presented positive clinical proof-of-concept data for an a-syn positron emission tomography (PET) tracer, as an imaging agent to identify MSA patients (Capotosti F.; Discovery of [¹⁸F] ACI-12589, a novel and promising PET-tracer for alpha-synuclein; Oral presentation; ADPD 2022 International Conference; Barcelona, Spain; Mar. 18, 2022; Smith R.; Initial scans using [¹⁸F] ACI-12589, a novel PET-tracer for alpha-synuclein; Oral presentation; ADPD 2022 International Conference; Barcelona, Spain; Mar. 18, 2022).

There is a clear need to find molecular probes with high alpha-synuclein selectivity which recognize and bind to the pathological alpha-synuclein. In order to minimize background signal interference resulting from non-specific off-target binding and to reduce dosing requirements, alpha-synuclein imaging compounds should bind with high affinity and selectivity to their target.

For imaging of alpha-synuclein aggregates associated with neurological diseases such as Parkinson's Disease or multiple system atrophy (MSA), imaging compounds need to penetrate the blood brain barrier and pass into the relevant regions of the brain. For targeting intracellular amyloid-like inclusions such as alpha-synuclein, cell permeability is a further requirement of imaging compounds. A further prerequisite in order to avoid unnecessary accumulation of the compound which may result in increased risk of unwanted side-effects is a fast compound wash-out from the brain (or other targeting organ).

WO 2011/128455 refers to specific compounds which are suitable for treating disorders associated with amyloid proteins or amyloid-like proteins. US 2012/0302755 relates to certain imaging agents for detecting neurological dysfunction. Further compounds for the diagnosis of neurodegenerative disorders on the olfactory epithelium are discussed in WO 2012/037928.

WO 2010/063701 refers to a certain in vivo imaging agent for use in a method to determine the presence of, or susceptibility to, Parkinson's disease, wherein the in vivo imaging agent comprises an alpha-synuclein binder labelled with an in vivo imaging moiety, and wherein the in vivo imaging agent binds to alpha-synuclein with a binding affinity.

US 2014/0142089 relates to a method for preventing or treating a degenerative brain disease, the method comprising administering to a subject in need thereof an effective amount of a pharmaceutical composition comprising a specific compound, a pharmaceutically acceptable salt, an isomer, a solvate, a hydrate, and a combination thereof.

WO 2009/155017 describes aryl or heteroaryl substituted azabenzoxazole derivatives, which are stated to be useful as tracers in positron emission tomography (PET) imaging to study amyloid deposits in the brain in vivo to allow diagnosis of Alzheimer's disease.

WO 2016/033445 refers to a specific compound for imaging huntingtin protein.

WO 2017/153601, WO 2019/234243 and WO 2021/224489 refer to bicyclic compounds for imaging alpha-synuclein aggregates.

There remains a need for a new class of imaging compounds that bind with reasonably high affinity to alpha-synuclein.

### SUMMARY OF THE INVENTION

The present invention provides compounds that can be employed in diagnosing a disease, disorder or abnormality associated with alpha-synuclein aggregates such as Parkinson's disease or MSA, prognosing such a disease, disorder or abnormality, and monitoring the progression of such a disease, disorder or abnormality. In particular, the compounds should be suitable for determining a predisposition to such a disease, disorder or abnormality, monitoring the progression of the disease, disorder or abnormality, or predicting the responsiveness of a patient who is suffering from such a disease, disorder or abnormality to the treatment with a certain medicament. Furthermore, the compounds should be suitable for positron emission tomography (PET) imaging of a disease, disorder or abnormality associated with alpha-synuclein aggregates and/or detecting and optionally quantifying alpha-synuclein aggregates.

Various embodiments of the invention are described herein.

Within a certain aspect, provided herein is a compound of formula (I):

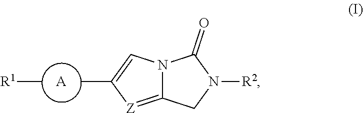

or a detectably labelled compound, stereoisomer, racemic mixture, pharmaceutically acceptable salt, hydrate, or solvate thereof, wherein

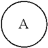

is a 6-membered heteroaryl, which is optionally substituted with at least one halo, haloC₁-C₄alkyl, haloC₁-C₄alkoxy, C₁-C₄alkoxy, or C₁-C₄alkyl;
R¹ is a 4- to 6-membered heterocyclyl which is optionally substituted with at least one halo; or
R¹ is halo, haloC₁-C₄alkyl, haloC₁-C₄alkoxy, C₁-C₄alkoxy, or C₁-C₄alkyl; or
R¹ is —NH—C₃-C₆cycloalkyl or —C₃-C₆cycloalkyl, wherein the C₃-C₆cycloalkyl of —NH—C₃-C₆cycloalkyl or —C₃-C₆cycloalkyl is optionally substituted with at least one halo; R² is a 5-membered or 6-membered heteroaryl, optionally substituted with 1 or 2 substituents independently selected from haloC₁-C₄alkyl, haloC₁-C₄alkoxy, C₁-C₄alkoxy, and C₁-C₄alkyl; and
Z is CH or N.

In another aspect, the invention is also directed to a compound having the following subformula (Ia)

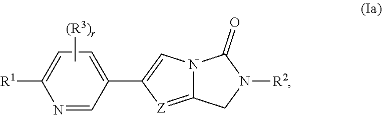

or a detectably labelled compound, stereoisomer, racemic mixture, pharmaceutically acceptable salt, hydrate, or solvate thereof,
wherein R³ is halo, haloC₁-C₄alkyl, haloC₁-C₄alkoxy, C₁-C₄alkoxy, or C₁-C₄alkyl, preferably R³ is halo, or C₁-C₄alkyl; and
r is 0, 1 or 2, preferably 0.

In one aspect, the present invention provides a diagnostic composition comprising a compound of formula (I), and optionally at least one pharmaceutically acceptable excipient, carrier, diluent and/or adjuvant.

In one aspect, the present invention provides a compound of formula (I), or a diagnostic composition as defined herein, which can be used in the imaging of alpha-synuclein aggregates.

In another aspect, the compound of formula (I), or the diagnostic composition can be for use in positron emission tomography imaging of alpha-synuclein aggregates.

In another aspect, the compound of formula (I) or the diagnostic composition, as defined herein, can be for use for in vitro imaging, ex vivo imaging, or in vivo imaging, preferably the use is for in vivo imaging, more preferably the use is for brain imaging.

In yet another aspect, the compound of formula (I) or the diagnostic composition, as defined herein, can be for use in diagnosis.

In a further aspect, the present invention refers to a method of diagnosing a disease, disorder or abnormality associated with alpha-synuclein aggregates, in a subject, the method comprising the steps:
(a) Administering a compound of formula (I), or a diagnostic composition which comprises a compound of formula (I), as defined herein, to the subject;
(b) Allowing the compound to bind to the alpha-synuclein aggregates; and
(c) Detecting the compound bound to the alpha-synuclein aggregates.

In another aspect, the present invention refers to a method of positron emission tomography (PET) imaging of alpha-synuclein aggregates in a tissue of a subject, the method comprising the steps:
(a) Administering a compound of formula (I), or a diagnostic composition which comprises a compound of formula (I), as defined herein to the subject;
(b) Allowing the compound to bind to the alpha-synuclein aggregates; and
(c) Detecting the compound bound to the alpha-synuclein aggregates by collecting a positron emission tomography (PET) image of the tissue of the subject.

In a further aspect, the present invention is directed to a method for the detection and optionally quantification of alpha-synuclein aggregates in a tissue of a subject, the method comprising the steps:
(a) Bringing a sample or a specific body part or body area suspected to contain alpha-synuclein aggregates into contact with a compound of formula (I), or a diagnostic composition which comprises a compound of formula (I), as defined herein;
(b) Allowing the compound to bind to the alpha-synuclein aggregates;
(c) Detecting the compound bound to the alpha-synuclein aggregates; and
(d) Optionally quantifying the amount of the compound bound to the alpha-synuclein aggregates.

The present invention is also directed to a method of collecting data for the diagnosis of a disease, disorder or abnormality associated with alpha-synuclein aggregates, wherein the method comprises the steps:
(a) Bringing a sample or a specific body part or body area suspected to contain alpha-synuclein aggregates into contact with a compound of the formula (I), or a diagnostic composition which comprises a compound of formula (I), as defined herein;
(b) Allowing the compound to bind to the alpha-synuclein aggregates;
(c) Detecting the compound bound to the alpha-synuclein aggregates; and
(d) Optionally correlating the presence or absence of the compound bound to the alpha-synuclein aggregates with the presence or absence of the alpha-synuclein aggregates in the sample or specific body part or body area.

The present invention also refers to a method of collecting data for determining a predisposition to a disease, disorder or abnormality associated with alpha-synuclein aggregates, the method comprising the steps:
(a) Bringing a sample or a specific body part or body area suspected to contain alpha-synuclein aggregates into contact with a compound of the formula (I), or a diagnostic composition which comprises a compound of formula (I), as defined herein;
(b) Allowing the compound to bind to the alpha-synuclein aggregates;
(c) Detecting the compound bound to the alpha-synuclein aggregates; and
(d) Optionally correlating the presence or absence of the compound bound to the alpha-synuclein aggregates with the presence or absence of the alpha-synuclein aggregates in the sample or specific body part or body area.

In a further aspect, the present invention also relates to a method of collecting data for prognosing a disease, disorder or abnormality associated with alpha-synuclein aggregates, wherein the method comprises the steps:
(a) Bringing a sample, a specific body part or body area suspected to contain alpha-synuclein aggregates into contact with a compound of the formula (I), or a diagnostic composition which comprises a compound of formula (I), as defined herein;
(b) Allowing the compound to bind to the alpha-synuclein aggregates;
(c) Detecting the compound bound to the alpha-synuclein aggregates;
(d) Optionally correlating the presence or absence of the compound bound to the alpha-synuclein aggregates with the presence or absence of the alpha-synuclein aggregates in the sample or specific body part or body area; and
(e) Optionally repeating steps (a) to (c) and, if present, optional step (d) at least one time.

In another aspect, the present invention is directed to a method of collecting data for monitoring the progression of a disease, disorder or abnormality associated with alpha-synuclein aggregates in a patient, the method comprising the steps:
(a) Bringing a sample, a specific body part or body area suspected to contain alpha-synuclein aggregates into contact with the compound of the formula (I), or a diagnostic composition which comprises a compound of formula (I), as defined herein;
(b) Allowing the compound to bind to the alpha-synuclein aggregates;
(c) Detecting the compound bound to the alpha-synuclein aggregates;
(d) Optionally correlating the presence or absence of the compound bound to the alpha-synuclein aggregates with the presence or absence of the alpha-synuclein aggregates in the sample or specific body part or body area; and
(e) Optionally repeating steps (a) to (c) and, if present, optional step (d) at least one time.

In a further aspect, the present invention relates to a method of collecting data for predicting responsiveness of a patient suffering from a disease, disorder or abnormality associated with alpha-synuclein aggregates to a medicament, the method comprising the steps:
(a) Bringing a sample, a specific body part or body area suspected to contain alpha-synuclein aggregates into contact with a compound of formula (I), or a diagnostic composition which comprises a compound of formula (I), as defined herein;
(b) Allowing the compound to bind to the alpha-synuclein aggregates;
(c) Detecting the compound bound to the alpha-synuclein aggregates;
(d) Optionally correlating the presence or absence of the compound bound to the alpha-synuclein aggregates with the presence or absence of the alpha-synuclein aggregates in the sample or specific body part or body area; and
(e) Optionally repeating steps (a) to (c) and, if present, optional step (d) at least one time.

In another aspect, the invention is further directed to a compound of formula (III-F):

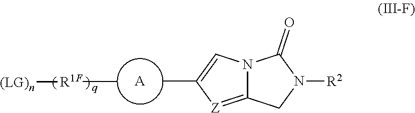

or a stereoisomer, racemic mixture, pharmaceutically acceptable salt, hydrate, or solvate thereof, wherein

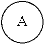

is a 6-membered heteroaryl, which is optionally substituted with at least one halo, haloC₁-C₄alkyl, haloC₁-C₄alkoxy, C₁-C₄alkoxy, or C₁-C₄alkyl;
R^{1F} is a 4- to 6-membered heterocyclyl; or
R^{1F} is C₁-C₄alkoxy, or C₁-C₄alkyl; or
R^{1F} is —NH—C₃-C₆cycloalkyl or C₃-C₆cycloalkyl;
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
Z is CH or N;
LG is a leaving group;
q is 0 or 1; and
n is at least 1 (e.g., 1, 2 or 3), preferably 1.

In another aspect, the invention is further directed to a compound of formula (I), wherein the compound is a detectably labelled compound of formula (I-F):

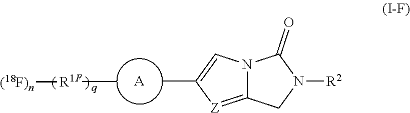

or a stereoisomer, racemic mixture, pharmaceutically acceptable salt, hydrate, or solvate thereof, wherein

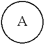

is a 6-membered heteroaryl, which is optionally substituted with at least one halo, haloC₁-C₄alkyl, haloC₁-C₄alkoxy, C₁-C₄alkoxy, or C₁-C₄alkyl;
R^{1F} is a 4- to 6-membered heterocyclyl; or
R^{1F} is C₁-C₄alkoxy, or C₁-C₄alkyl; or
R^{1F} is —NH—C₃-C₆cycloalkyl or —C₃-C₆cycloalkyl;
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
Z is CH or N;
q is 0 or 1; and
n is at least 1 (e.g., 1, 2 or 3), preferably 1.

In another aspect, the invention is further directed to compound of formula (III-H)

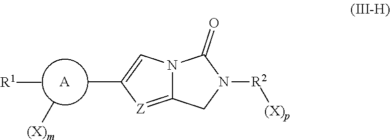

or a stereoisomer, racemic mixture, pharmaceutically acceptable salt, hydrate, or solvate thereof, wherein

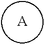

is a 6-membered heteroaryl, which is optionally substituted with at least one halo, haloC₁-C₄alkyl, haloC₁-C₄alkoxy, C₁-C₄alkoxy, or C₁-C₄alkyl;
R¹ is a 4- to 6-membered heterocyclyl which is optionally substituted with at least one halo; or
R¹ is halo, haloC₁-C₄alkyl, haloC₁-C₄alkoxy, C₁-C₄alkoxy, or C₁-C₄alkyl; or
R¹ is —NH—C₃-C₆cycloalkyl or —C₃-C₆cycloalkyl, wherein the C₃-C₆cycloalkyl of —NH—C₃-C₆cycloalkyl or —C₃-C₆cycloalkyl is optionally substituted with at least one halo;
R² is a 5-membered or 6-membered heteroaryl, optionally substituted with 1 or 2 substituents independently selected from haloC₁-C₄alkyl, haloC₁-C₄alkoxy, C₁-C₄alkoxy, and C₁-C₄alkyl;
Z is CH or N;
X is bromo, chloro or iodo;
m is 0, 1, 2 or 3;
p is 0, 1, 2 or 3; and
with the proviso that the compound of formula (III-H) comprises at least one X.

Another aspect of the invention is further directed to compound of formula (I-H)

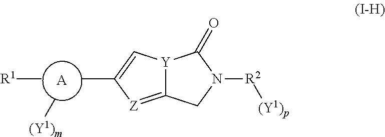

or a stereoisomer, racemic mixture, pharmaceutically acceptable salt, hydrate, or solvate thereof, wherein

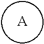

is a 6-membered heteroaryl, which is optionally substituted with at least one halo, haloC₁-C₄alkyl, haloC₁-C₄alkoxy, C₁-C₄alkoxy, or C₁-C₄alkyl;
R¹ is a 4- to 6-membered heterocyclyl which is optionally substituted with at least one halo; or
R¹ is halo, haloC₁-C₄alkyl, haloC₁-C₄alkoxy, C₁-C₄alkoxy, or C₁-C₄alkyl; or
R¹ is —NH—C₃-C₆cycloalkyl or —C₃-C₆cycloalkyl, wherein the C₃-C₆cycloalkyl of —NH—C₃-C₆cycloalkyl or —C₃-C₆cycloalkyl is optionally substituted with at least one halo;
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
Z is CH or N;
Y¹ is D, CD₃, T or CT₃;
m is 0, 1, 2 or 3;
p is 0, 1, 2 or 3; and
with the proviso that the compound of formula (I-H) comprises at least one D, CD₃, T or CT₃, wherein D is ²H (deuterium) and T is ³H (Tritium). In one embodiment, the compound of formula (I-H) comprises at least one T or CT₃. In one embodiment, the compound of formula (I-H) comprises at least one D or CD₃.

In another aspect, the invention is further directed to a method of preparing a compound of formula (I-F) by reacting a compound of formula (III-F) with a ¹⁸F-fluorinating agent (e.g., K¹⁸F, Rb¹⁸F, Cs¹⁸F, Na¹⁸F, tetra(C₁₋₆alkyl)ammonium salt of ¹⁸F, Kryptofix[222]¹⁸F, tetrabutylammonium [¹⁸F]fluoride or any other suitable agents) so that the Leaving Group (LG) is replaced by ¹⁸F.

In another aspect, the invention is further directed to a method of preparing a compound of formula (I-H) by reacting the compound of formula (III-H) with a ³H radiolabelling agent (e.g., tritium gas or any other suitable agents), so that X is replaced by T or CT₃.

In another aspect, the invention is further directed to a method of preparing a compound of formula (I-H) by reacting the compound of formula (III-H) with a ²H radiolabelling agent comprising D (e.g., D₂O, D4-methanol or any other suitable agents), preferably in the presence of a catalyst like Pd/C, so that X is replaced by D or CD₃ (D is deuterium).

In another aspect, the invention is further directed to the use of the compound of formula (I) as an in vitro analytical reference or an in vitro screening tool.

In another aspect, the invention is further directed to a test kit for detection and/or diagnosis of a disease, disorder or abnormality associated with alpha-synuclein aggregates, wherein the test kit comprises at least one compound of formula (I) as defined herein, preferably at least one detectably labelled compound, more preferably at least one compound of formula (I-F) or (I-H).

The invention is further directed to a kit for preparing a radiopharmaceutical preparation, wherein the kit comprises a sealed vial containing at least one compound of formula (III-F) or (III-H).

### Definitions

For the purpose of interpreting this specification, the following definitions will apply unless specified otherwise, and when appropriate, terms used in the singular will also include the plural and vice versa. It must also be noted that as used herein and in the appended claims, the singular forms “a”, “an” and “the” include plural referents unless the context clearly dictates otherwise. Thus, for example, reference to “the compound” includes reference to one or more compounds; and so forth.

The term “C₁-C₄alkyl” refers to a saturated straight or branched hydrocarbon chain consisting solely of carbon and hydrogen atoms, containing no unsaturation, having from one to four carbon atoms, and which is attached to the rest of the molecule by a single bond. Examples of suitable alkyl groups having 1 to 4 carbon atoms include, but are not limited to, methyl, ethyl, propyl, isopropyl, 1-methylethyl, n-butyl, t-butyl and isobutyl.

The term “C₁-C₄alkoxy” refers to a radical of the formula —ORa where Ra is a C₁-C₄alkyl radical as generally defined above. Examples of C₁-C₄alkoxy include, but are not limited to, methoxy, ethoxy, propoxy, isopropoxy, butoxy, and isobutoxy.

The term “halogenC₁-C₄alkyl” or “haloC₁-C₄alkyl” refer to a C₁-C₄alkyl radical as defined above, substituted with one or more (e.g., 1, 2 or 3, preferably 1 or 2, more preferably 1) halo radicals as defined below. Examples of “haloC₁-C₄alkyl” include, but are not limited to, trifluoromethyl, difluoromethyl, fluoromethyl, trichloromethyl, 2,2,2-trifluoroethyl, 1,3-dibromopropan-2-yl, 3-bromo-2-fluoropropyl and 1,4,4-trifluorobutan-2-yl.

The term “halogenC₁-C₄alkoxy” refers to a C₁-C₄alkoxy radical as defined above, substituted with one or more (e.g., 1, 2 or 3, preferably 1 or 2, more preferably 1) halo radicals as defined below.

Examples of “haloC₁-C₄alkoxy” include, but are not limited to, trifluoromethoxy, difluoromethoxy, fluoromethoxy, 2,2,2-trifluoroethoxy, 3,3,3-trifluoropropoxy, 4,4,4-trifluorobutoxy, 2,2-difluorobutoxy, and 4-bromobutoxy.

The term “heterocyclyl” refers to a stable 4- to 6-membered non-aromatic monocyclic ring radical which comprises 1 or 2 heteroatoms which are, e.g., selected from N, O or S. The heterocyclyl group can be unsaturated or saturated. The heterocyclyl radical may be bonded via a carbon atom or a heteroatom. Examples include, but are not limited to, azetidinyl, oxetanyl, pyrrolidinyl, pyrrolidyl, tetrahydrofuryl, tetrahydrothienyl, piperidyl, piperazinyl, tetrahydropyranyl, or morpholinyl, preferably azetidinyl, pyrrolidinyl, or piperidyl, more preferably pyrrolidinyl.

The term “heteroaryl” refers to a 5- or 6-membered aromatic monocyclic ring, which comprises 1, 2, or 3 heteroatoms independently selected from N, O and S. The heteroaryl radical may be bonded via a carbon atom or heteroatom selected from N, O and S. Examples of heteroaryl include, but are not limited to, thiopyranyl, dioxanyl, pyranyl, pyrazinyl, pyridazinyl, pyrimidyl, isothiazolyl, pyrazolyl, thiazolyl or pyridyl, with pyridyl, isothiazolyl, pyrazolyl, and thiazolyl being preferred, and pyridyl being the most preferred.

The term “Hal” or “halogen” or “Halo” refers to F, Cl, Br, and I. With respect to diagnostic and pharmaceutical applications, F (e.g., ¹⁹F and ¹⁸F) is particularly preferred.

The term “leaving group” (LG) as employed herein is any leaving group and means an atom or group of atoms that can be replaced by another atom or group of atoms. Examples are given e.g. in Synthesis (1982), p. 85-125, table 2, Carey and Sundberg, Organische Synthese, (1995), page 279-281, table 5.8; or Netscher, Recent Res. Dev. Org. Chem., 2003, 7, 71-83, schemes 1, 2, 10 and 15 and others). (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50, explicitly: scheme 4 pp. 25, scheme 5 pp 28, table 4 pp 30, Figure 7 pp 33). Preferably, the “leaving group” (LG) is selected from halogen (bromo, chloro, iodo), nitro, C₁-C₄alkylsulfonate and C₆-C₁₀arylsulfonate, wherein the C₆-C₁₀arylsulfonate can be optionally substituted with —CH₃ or —NO₂.

Unless specified otherwise, the term “compound of the invention” refers to a compound of formula (I), or of subformulae thereof (e.g. (I-F), (I-H*), (I-H)), or a detectably labelled compound, stereoisomer (including diastereomeric mixtures and individual diastereomer, enantiomeric mixture and single enantiomer, mixture of conformers and single conformer), racemic mixture, pharmaceutically acceptable salt, hydrate, or solvate thereof. It is understood that every reference to a compound of formula (I) also covers the subformulae thereof (e.g. (I-F), (I-H*), (I-H)). The compounds of the formulae (III-F) and (III-H) will be referred to as the precursors of the compounds of the present invention.

Compounds of the present invention and their precursors having one or more optically active carbons can exist as racemates and racemic mixtures, stereoisomers (including diastereomeric mixtures and individual diastereomers, enantiomeric mixtures and single enantiomers, mixtures of conformers and single conformers), tautomers, atropisomers, and rotamers. All isomeric forms are included in the present invention.

“Pharmaceutically acceptable salts” are defined as derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. The pharmaceutically acceptable salts of the compounds of the present invention and their precursors can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts can be found in Remington's Pharmaceutical Sciences, 18^{th} ed., Mack Publishing Company, Easton, PA, 1990, p. 1445, the disclosure of which is hereby incorporated by reference.

“Pharmaceutically acceptable” is defined as those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

“Solvates” can be formed from the compound of the present invention and any suitable pharmaceutically acceptable solvent. Examples include C₁₋₄ alcohols (such as methanol or ethanol).

The patients or subjects in the present invention are typically animals, particularly mammals, more particularly humans.

Alpha-synuclein aggregates are multimeric beta-sheet rich assemblies of alpha-synuclein monomers that can form either soluble oligomers or soluble/insoluble protofibrils or mature fibrils which coalesce into intracellular deposits detected as a range of Lewy pathologies in Parkinson's disease and other synucleinopathies. Alpha-synuclein aggregates that are composing Lewy pathologies can be detected as having the following morphologies: Lewy bodies, Lewy neurites, premature Lewy bodies or pale bodies, perikaryal deposits with diffuse, granular, punctate or pleomorphic patterns. Moreover, alpha-synuclein aggregates are the major component of intracellular fibrillary inclusions detected in oligodendrocytes (also referred to as glial cytoplasmic inclusions) and in neuronal somata, axons and nuclei (referred to as neuronal cytoplasmic inclusions) that are the histological hallmarks of multiple system atrophy. Alpha-synuclein aggregates in Lewy pathologies often display substantial increase in post-translational modifications such as phosphorylation, ubiquitination, nitration, and truncation.

Lewy bodies are abnormal aggregates of protein that develop inside nerve cells in Parkinson's disease (PD), Lewy body dementia and other synucleinopathies. Lewy bodies appear as spherical masses that displace other cell components. Morphologically, Lewy bodies can be classified as being brainstem or cortical type. Classic brainstem Lewy bodies are eosinophilic cytoplasmic inclusions consisting of a dense core surrounded by a halo of 5-10-nm-wide radiating fibrils, the primary structural component of which is alpha-synuclein; cortical Lewy bodies differ by lacking a halo. The presence of Lewy bodies is a hallmark of Parkinson's disease.

Lewy neurites are abnormal neuronal processes in diseased neurons, containing granular material, abnormal alpha-synuclein (a-syn) filaments similar to those found in Lewy bodies, dot-like, varicose structures and axonal spheroids. Like Lewy bodies, Lewy neurites are a feature of α-synucleinopathies such as dementia with Lewy bodies and Parkinson's disease.

Glial cytoplasmic inclusions (GCIs or Papp-Lantos bodies) are argyrophilic cytoplasmic aggregates in oligodendroglial cells composed of filamentous alpha-synuclein. Morphologically appear as triangles, half-moon or sickle shapes. In MSA, besides GCIs, inclusions composed of alpha-synuclein filaments are detected in neurons in the cytoplasm or beneath the nuclear membrane termed neuronal cytoplasmic inclusions and neuronal nuclear inclusions respectively. GCIs are recognized as the defining morphological feature of MSA; their widespread distribution is a criterion for the definite post-mortem neuropathological diagnosis of MSA.

The terms “disease”, “disorder” or “abnormality” are used interchangeably herein.

The compounds of formula (I) can bind to alpha-synuclein aggregates. The type of bonding with the compounds of formula (I) has not been elucidated and any type of bonding is covered by the present invention. The wording “compound bound to the alpha-synuclein aggregates” and the like are used interchangeably herein and are not considered to be limited to any specific type of bonding.

The preferred definitions given in the “Definition”-section apply to all of the embodiments described below unless stated otherwise. Various embodiments of the invention are described herein, it will be recognized that features specified in each embodiment may be combined with other specified features to provide further embodiments of the present invention.

### DETAILED DESCRIPTION OF THE INVENTION

### Compounds of the Invention

The compounds of the present invention and their precursors are described in the following. It is to be understood that all possible combinations of the following definitions are also envisaged. It is also understood that all of the embodiments and preferred embodiments which are given with respect to the formula (I) apply analogously to the formulae (III-F), (I-F), (III-H), (I-H) and (I-H*), etc. and vice versa. It is also understood that the preferred embodiments of which are given with respect to the formula (III-F) apply analogously to the formula (I-F) and vice versa. It is also understood that the preferred embodiments of which are given with respect to the formula (III-H) apply analogously to the formula (I-H) and (I-H*), respectively, and vice versa.

The present invention relates to a compound of formula (I):

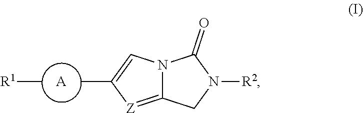

or a detectably labelled compound, stereoisomer, racemic mixture, pharmaceutically acceptable salt, hydrate, or solvate thereof, wherein

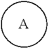

is a 6-membered heteroaryl, which is optionally substituted with at least one halo, haloC₁-C₄alkyl, haloC₁-C₄alkoxy, C₁-C₄alkoxy, or C₁-C₄alkyl; if

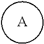

is substituted,

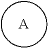

is substituted with 1, 2 or 3, preferably 1 or 2, more preferably with 1 halo, haloC₁-C₄alkyl, haloC₁-C₄alkoxy, C₁-C₄alkoxy, or C₁-C₄alkyl;
R¹ is a 4- to 6-membered heterocyclyl which is optionally substituted with at least one halo; or
R¹ is halo, haloC₁-C₄alkyl, haloC₁-C₄alkoxy, C₁-C₄alkoxy, or C₁-C₄alkyl; or
R¹ is —NH—C₃-C₆cycloalkyl or —C₃-C₆cycloalkyl, wherein the C₃-C₆cycloalkyl of —NH—C₃-C₆cycloalkyl or —C₃-C₆cycloalkyl is optionally substituted with at least one halo;
R² is a 5-membered or 6-membered heteroaryl, optionally substituted with 1 or 2 substituents independently selected from haloC₁-C₄alkyl, haloC₁-C₄alkoxy, C₁-C₄alkoxy, and C₁-C₄alkyl; and
Z is CH or N.

In one embodiment,

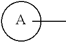

is selected from the group consisting of

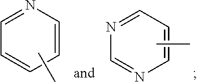

which can be attached at any available position to the heterocycle;
and wherein

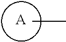

can be optionally substituted by one or more substituents selected from halo; haloC₁-C₄alkyl, haloC₁-C₄alkoxy, C₁-C₄alkoxy, or C₁-C₄alkyl.

In a preferred embodiment,

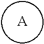

a 6-membered heteroaryl comprising at least one N, preferably

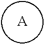

is pyridyl, most preferably

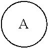

is pyridyl and is not substituted.

The optional substituent of

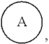

if present, is preferably halo or C₁-C₄alkyl.

Preferably,

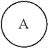

is unsubstituted.

In another embodiment, the invention provides a compound having the formula (Ia):

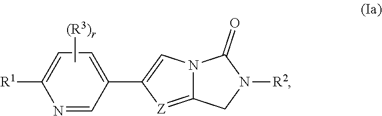

or a detectably labelled compound, stereoisomer, racemic mixture, pharmaceutically acceptable salt, hydrate, or solvate thereof,
wherein Z, R¹ and R² are as defined above, and R³ is halo, haloC₁-C₄alkyl, haloC₁-C₄alkoxy, C₁-C₄alkoxy, or C₁-C₄alkyl, preferably R³ is halo or haloC₁-C₄alkyl; and
r is 0, 1, or 2; preferably 0.

In one embodiment, R¹ is a 4- to 6-membered heterocyclyl which is optionally substituted with at least one halo (e.g., 1 to 3, preferably 1 or 2, more preferably 1 halo). In a preferred embodiment, R¹ is a 4- to 6-membered heterocyclyl which is substituted with at least one halo. Preferably, the heterocyclyl is substituted with at least one halo, more preferably with one or two halo, even more preferably with one halo.

In another embodiment, R¹ is halo, haloC₁-C₄alkyl, haloC₁-C₄alkoxy, C₁-C₄alkoxy, or C₁-C₄alkyl. Preferably, R¹ is haloC₁-C₂alkoxy, more preferably —OCH₂—CH₂—F.

In an embodiment, R¹ is selected from the following:

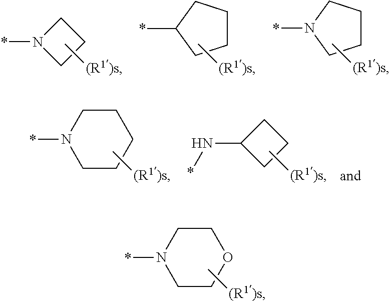

wherein R^{1′} is independently halo; and s=0, 1, 2 or 3.

In one preferred embodiment, R¹ is a 4- to 6-membered heterocyclyl selected from the following:

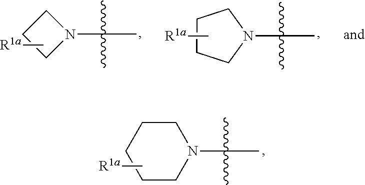

wherein R^{1a} is F.

Preferably, R¹ is a 4- or 5-membered heterocyclyl selected from the following:

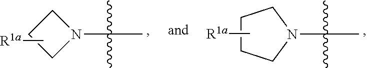

wherein R^{1a} is F.

In another embodiment, R¹ is halo, haloC₁-C₄alkyl, haloC₁-C₄alkoxy, C₁-C₄alkoxy, or C₁-C₄alkyl. Preferably, R¹ is haloC₁-C₄alkyl or halo, more preferably R¹ is —O—CH₂—CH₂—F or F. More preferably, R¹ is halo, even more preferably R¹ is F.

In yet another most preferred embodiment R¹ is a 5-membered heterocyclyl which is:

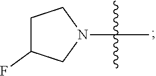 [SMILES: *N1CCC(F)C1]

or R¹ is F.

In a further embodiment, R¹ is —NH—C₃-C₆cycloalkyl or C₃-C₆cycloalkyl, wherein the C₃-C₆cycloalkyl of —NH—C₃-C₆cycloalkyl or —C₃-C₆cycloalkyl is optionally substituted with at least one halo.

In each of the embodiments, F is preferably ¹⁹F or ¹⁸F.

In one embodiment, R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl.

In a preferred embodiment, R² is a 5-membered or 6-membered heteroaryl selected from the following:

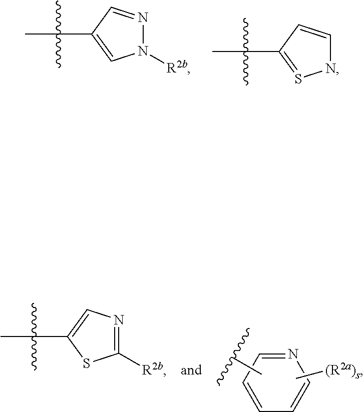

wherein
R^{2a} is selected from haloC₁-C₄alkyl, haloC₁-C₄alkoxy, C₁-C₄alkoxy, and C₁-C₄alkyl;
R^{2b} is selected from H, haloC₁-C₄alkyl, haloC₁-C₄alkoxy, C₁-C₄alkoxy, and C₁-C₄alkyl; and
s is 0, 1 or 2 (preferably s is 0 or 1, more preferably 0).

Preferably, R² is a 5-membered or 6-membered heteroaryl selected from the following:

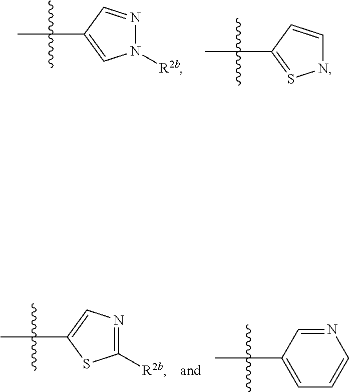

wherein
R^{2b} is selected from H or C₁-C₄alkyl.

More preferably, R² is a 5-membered or 6-membered heteroaryl selected from the following:

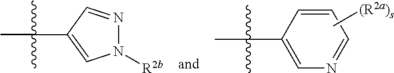

wherein
R^{2a} is independently selected from haloC₁-C₄alkyl, haloC₁-C₄alkoxy, C₁-C₄alkoxy, and C₁-C₄alkyl;
R^{2b} is selected from H, haloC₁-C₄alkyl and C₁-C₄alkyl, wherein haloC₁-C₄alkyl and C₁-C₄alkyl; and
s is 0, 1 or 2 (preferably 0 or 1, more preferably 0).

Even more preferably, R² is a 5-membered or 6-membered heteroaryl selected from the following:

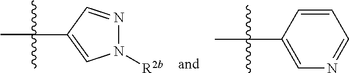

wherein
R^{2b} is selected from C₁-C₄alkyl.

In a most preferred embodiment, R² is a 6-membered unsubstituted heteroaryl or R² is 5-membered heteroaryl substituted with C₁-C₄alkyl.

In one embodiment, the present invention provides a compound of formula (I), wherein the compound is selected from

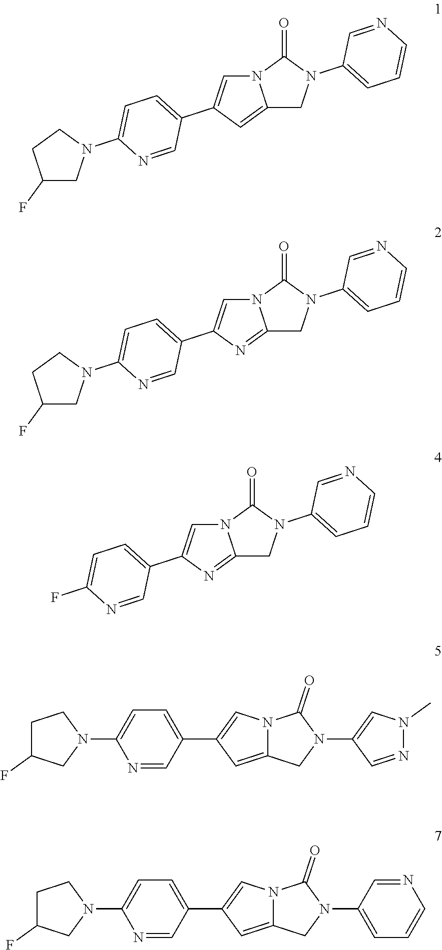 [SMILES: O=C1N(c2cccnc2)Cc2cc(-c3ccc(N4CCC(F)C4)nc3)cn21 ; O=C1N(c2cccnc2)Cc2nc(-c3ccc(N4CCC(F)C4)nc3)cn21 ; O=C1N(c2cccnc2)Cc2nc(-c3ccc(F)nc3)cn21 ; Cn1cc(N2Cc3cc(-c4ccc(N5CCC(F)C5)nc4)cn3C2=O)cn1 ; O=C1N(c2cccnc2)Cc2cc(-c3ccc(N4CCC(F)C4)nc3)cn21]

or a detectably labelled compound, stereoisomer, pharmaceutically acceptable salt, hydrate, or solvate thereof.

In one embodiment, the present invention provides a compound of formula (I), wherein the compound is selected from

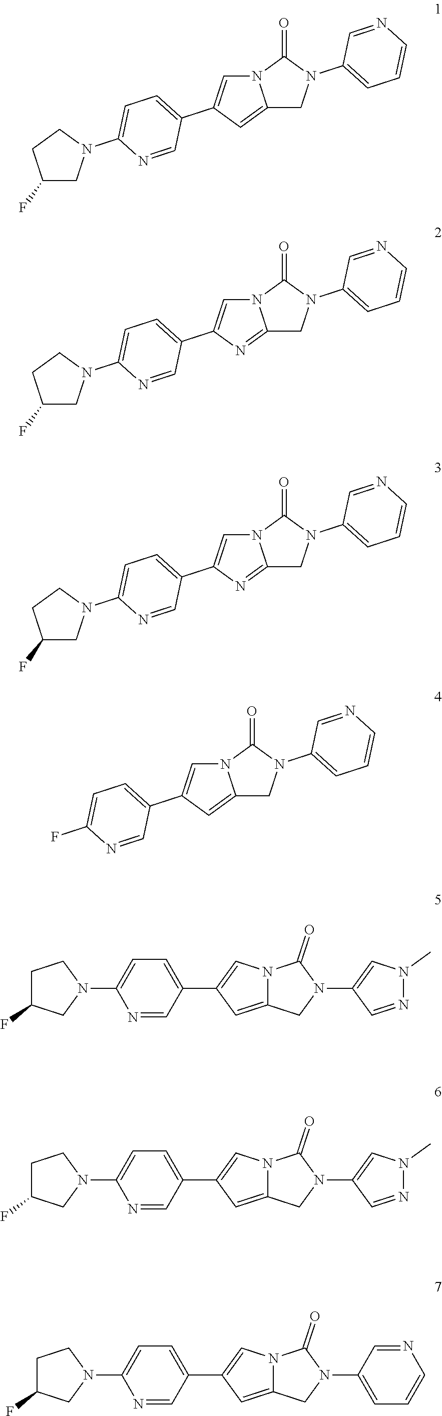

or a detectably labelled compound, pharmaceutically acceptable salt, hydrate, or solvate thereof.

In one embodiment, the present invention provides a compound of formula (I) which is a detectably labelled compound. The detectable label can be a radioisotope. In one embodiment, the compound of formula (I) comprises at least one radioisotope. Preferably, the detectable label is selected from ¹⁸F, ²H and ³H. Most preferably, the radioisotope is selected from ¹⁸F and ³H.

In one embodiment, the present invention provides a compound of formula (I) wherein R¹ is

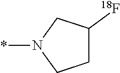 [SMILES: *N1CCC([18F])C1]

or ¹⁸F, with

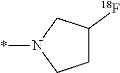 [SMILES: *N1CCC([18F])C1]

being preferred.

In one embodiment the present invention provides a compound of formula (I), wherein the compound is a detectably labelled compound of formula (I-F):

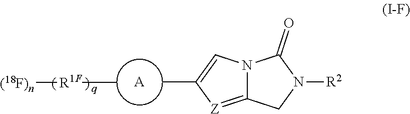

or a stereoisomer, racemic mixture, pharmaceutically acceptable salt, hydrate, or solvate thereof, wherein

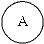

is a 6-membered heteroaryl, which is optionally substituted with at least one halo, haloC₁-C₄alkyl, haloC₁-C₄alkoxy, C₁-C₄alkoxy, or C₁-C₄alkyl;
R^{1F} is a 4- to 6-membered heterocyclyl; or
R^{1F} is C₁-C₄alkoxy, or C₁-C₄alkyl; and
R^{1F} is —NH—C₃-C₆cycloalkyl or —C₃-C₆cycloalkyl;
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
Z is CH or N;
q is 0 or 1; and
n is at least 1 (e.g., n is 0, 1, 2 or 3), preferably 1.

In one embodiment, R^{1F} is —NH—C₃-C₆cycloalkyl, C₃-C₆cycloalkyl, C₁-C₄alkoxy, or C₁-C₄alkyl or heterocyclyl. Preferably —R^{1F}—(¹⁸F)ₙ is selected from the following:

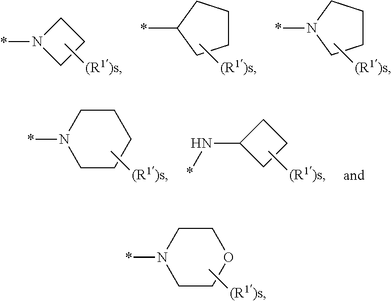

wherein R^{1′} is ¹⁸F; and s=1, 2 or 3, preferably s=1.

In a preferred embodiment, —R^{1F}—(¹⁸F)ₙ is selected from the following:

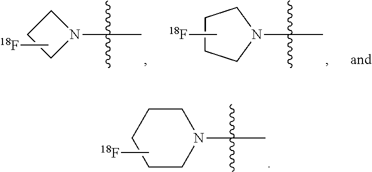

More preferably, —R^{1F}—(¹⁸F)ₙ is selected from the following:

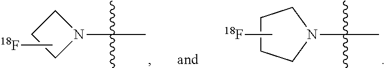

Even more preferably, —R^{1F}—(¹⁸F)ₙ is:

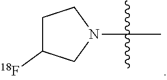 [SMILES: *N1CCC([18F])C1]

In another embodiment, —R^{1F}—(¹⁸F)ₙ is haloC₁-C₄alkoxy such as —O—CH₂—CH₂—¹⁸F.

In another preferred embodiment, q is 0, i.e., R^{1F}—(¹⁸F)ₙ is ¹⁸F.

The detectably labelled compound of formula (I-F) comprises at least one ¹⁸F. Preferably, the detectably labelled compound of formula (I-F) comprises one ¹⁸F.

In one embodiment, the present invention provides a compound of formula (I), wherein the compound is a compound of formula (I-H*)

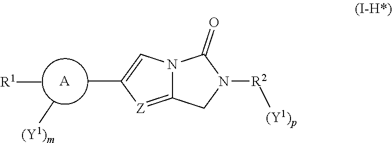

or a stereoisomer, racemic mixture, pharmaceutically acceptable salt, hydrate, or solvate thereof, wherein

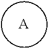

is a 6-membered heteroaryl, which is optionally substituted with at least one halo; haloC₁-C₄alkyl, haloC₁-C₄alkoxy, C₁-C₄alkoxy, or C₁-C₄alkyl; and
R¹ is a 4- to 6-membered heterocyclyl which is optionally substituted with at least one halo; or
R¹ is halo, haloC₁-C₄alkyl, haloC₁-C₄alkoxy, C₁-C₄alkoxy, or C₁-C₄alkyl; or
R¹ is —NH—C₃-C₆cycloalkyl or —C₃-C₆cycloalkyl, wherein the C₃-C₆cycloalkyl of —NH—C₃-C₆cycloalkyl or —C₃-C₆cycloalkyl is optionally substituted with at least one halo; and
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl; and
Z is CH or N; and
Y¹ is D, CD₃, T or CT₃;
m is 0, 1, 2 or 3; and
p is 0, 1, 2 or 3; and
with the proviso that the compound of formula (I-H*) comprises at least one ²H (deuterium “D”) or ³H (Tritium “T”), preferably 1, 2, or 3 D or T, even more preferably 2 or 3 D or T. Preferably, the compound of formula (I-H*) comprises at least one ³H (Tritium “T”), preferably 1, 2, or 3 T, even more preferably 2 or 3 T. The ³H can be present as T or as —CT₃. The ²H can be present as D or as —CD₃.

In a preferred embodiment, the compound is a detectably labelled compound of formula (I-H)

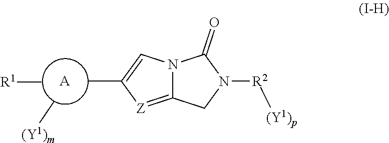

or a stereoisomer, racemic mixture, pharmaceutically acceptable salt, hydrate, or solvate thereof, wherein

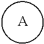

is a 6-membered heteroaryl, which is optionally substituted with at least one halo, haloC₁-C₄alkyl, haloC₁-C₄alkoxy, C₁-C₄alkoxy, or C₁-C₄alkyl;
R¹ is a 4- to 6-membered heterocyclyl which is optionally substituted with at least one halo; or
R¹ is halo, haloC₁-C₄alkyl, haloC₁-C₄alkoxy, C₁-C₄alkoxy, or C₁-C₄alkyl; or
R¹ is —NH—C₃-C₆cycloalkyl or —C₃-C₆cycloalkyl, wherein the C₃-C₆cycloalkyl of —NH—C₃-C₆cycloalkyl or —C₃-C₆cycloalkyl is optionally substituted with at least one halo; and
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
Z is CH or N;
Y¹ is D, CD₃, T or CT₃;
m is 0, 1, 2 or 3;
p is 0, 1, 2 or 3; and
with the proviso that the compound of formula (I-H) comprises at least one D, CD₃, T or CT₃, wherein D is ²H (Deuterium) and T is ³H (Tritium). In one embodiment, the compound of formula (I-H) comprises at least one D or CD₃. In another embodiment, the compound of formula (I-H) comprises at least one T or CT₃.

It is understood that the deuterium or tritium can present at any available position at which a hydrogen is present. For instance, in the group R² deuterium or tritium can be present either directly bound to the 5-membered or 6-membered heteroaryl (such as in the form of D or T) or can be present in the haloC₁-C₄alkyl, haloC₁-C₄alkoxy, C₁-C₄alkoxy, and C₁-C₄alkyl (such as in the form of CD₃ or CT₃). In the 4- to 6-membered heterocyclyl of R¹, deuterium or tritium can be, e.g., directly bound to the 4- to 6-membered heterocyclyl.

In one embodiment,

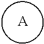

is a 6-membered heteroaryl optionally substituted with one halo or C₁-C₄alkyl and m is 1, 2 or 3, preferably 1 or 2.

More preferably,

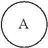

is unsubstituted.

In one embodiment, R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl, and p is 1, 2 or 3, preferably 1.

Preferably, R² is a 5-membered or 6-membered heteroaryl selected from the following:

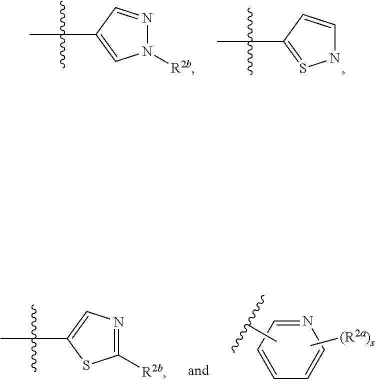

wherein
R^{2a} is independently selected from T, haloC₁-C₄alkyl, haloC₁-C₄alkoxy, C₁-C₄alkoxy, and C₁-C₄alkyl (e.g., CT₃);
R^{2b} is selected from H, T, haloC₁-C₄alkyl and C₁-C₄alkyl, wherein haloC₁-C₄alkyl and C₁-C₄alkyl optionally comprise one or more T (preferably R^{2b} is selected from T or CT₃); and
s is 0, 1 or 2 (preferably 0 or 1, more preferably 0).

Preferably, R² is a 5-membered or 6-membered heteroaryl selected from the following:

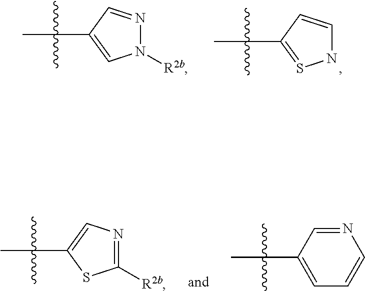

wherein
R^{2b} is selected from H, T or C₁-C₄alkyl (e.g., CT₃).

More preferably, R² is a 5-membered or 6-membered heteroaryl selected from the following:

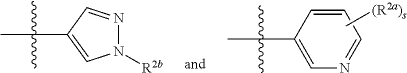

wherein
R^{2a} is independently selected from T, haloC₁-C₄alkyl, haloC₁-C₄alkoxy, C₁-C₄alkoxy, and C₁-C₄alkyl (e.g., CT₃);
R^{2b} is selected from H, T, haloC₁-C₄alkyl and C₁-C₄alkyl, wherein haloC₁-C₄alkyl and C₁-C₄alkyl optionally comprise one or more T (preferably R^{2b} is selected from T or CT₃); and
s is 0, 1 or 2 (preferably 0).

Even more preferably, R² is a 5-membered or 6-membered heteroaryl selected from the following:

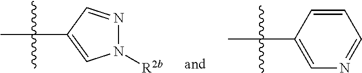

wherein
R^{2b} is selected from C₁-C₄alkyl, wherein C₁-C₄alkyl optionally comprise one or more T.

Preferably, R^{2a} is -T, —OCH₃, —CH₃, or —H; and R^{2b} is selected from —H, -T or —CT₃.

In a preferred embodiment, the detectably labelled compound of formula (I-H*) or (I-H) comprises one, two or three T. Preferably, the detectably labelled compound of formula (I-H*) or (I-H) comprises one T. In another embodiment, the detectably labelled compound of formula (I-H*) or (I-H) comprises two T. In another embodiment, the detectably labelled compound of formula (I-H*) or (I-H) comprises three T such as —CT₃.

In another embodiment, R² is a 5-membered or 6-membered heteroaryl selected from the following:

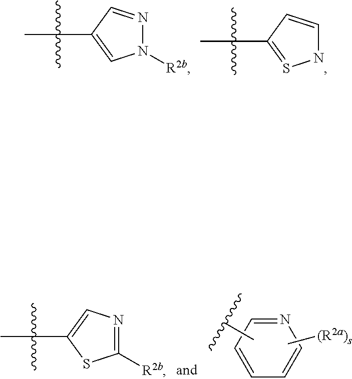

wherein
R^{2a} is independently selected from D, haloC₁-C₄alkyl, haloC₁-C₄alkoxy, C₁-C₄alkoxy, and C₁-C₄alkyl (e.g., CD₃);
R^{2b} is selected from H, D, haloC₁-C₄alkyl and C₁-C₄alkyl, wherein haloC₁-C₄alkyl and C₁-C₄alkyl optionally comprise one or more D (preferably R^{2b} is selected from D or CD₃); and
s is 0, 1 or 2 (preferably 0).

More preferably, R² is a 5-membered or 6-membered heteroaryl selected from the following:

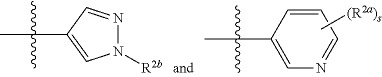

wherein
R^{2a} is independently selected from D, haloC₁-C₄alkyl, haloC₁-C₄alkoxy, C₁-C₄alkoxy, and C₁-C₄alkyl (e.g., CD₃);
R^{2b} is selected from H, D, haloC₁-C₄alkyl and C₁-C₄alkyl, wherein haloC₁-C₄alkyl and C₁-C₄alkyl optionally comprise one or more D (preferably R^{2b} is selected from D or CD₃); and
s is 0, 1 or 2 (preferably 0).

Even more preferably, R² is a 5-membered or 6-membered heteroaryl selected from the following:

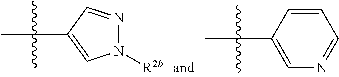

wherein
R^{2b} is selected from C₁-C₄alkyl, wherein C₁-C₄alkyl optionally comprise one or more D.

Preferably, R^{2a} is -D, —OCH₃, —CH₃, or —H; and R^{2b} is selected from —H, -D or —CD₃.

In one embodiment, the detectably labelled compound of formula (I-H*) or (I-H) comprises one, two or three D. Preferably, the detectably labelled compound of formula (I-H*) or (I-H) comprises one D. In another embodiment, the detectably labelled compound of formula (I-H*) or (I-H) comprises two D. In another embodiment, the detectably labelled compound of formula (I-H*) or (I-H) comprises three D such as —CD₃.

In another embodiment, the invention provides a detectably labelled compound of formula (I-H*) or (I-H) wherein ³H Tritium (“T”) can be replaced by ²H Deuterium (“D”). The deuterated compound can be prepared by reacting a compound of formula (III-H) with a ²H radiolabelling agent.

The compounds of the present invention and their precursors can be detectably labelled. The type of the label is not specifically limited and will depend on the detection method chosen. Examples of possible labels include isotopes such as radionuclides, positron emitters, and gamma emitters, preferably the detectable label is a radioisotope. With respect to the detectably labelled compounds of the present invention and their precursors which include a radioisotope, a positron emitter, or a gamma emitter, it is to be understood that the radioisotope, positron emitter, or gamma emitter is to be present in an amount which is not identical to the natural amount of the respective radioisotope, positron emitter, or gamma emitter. Furthermore, the employed amount should allow detection thereof by the chosen detection method. Examples of suitable isotopes such as radionuclides, positron emitters and gamma emitters include ²H, ³H, ¹¹C, ¹³N, ¹⁵O, and ¹⁸F, more preferably ²H, ³H and ¹⁸F.

¹⁸F-labelled compounds are particularly suitable for imaging applications such as PET. The corresponding compounds which include fluorine having a natural ¹⁹F isotope are also of particular interest as they can be used as analytical standards and references during manufacturing, quality control, release, and clinical use of their ¹⁸F-analogs.

Further, substitution with isotopes such as deuterium, i.e. ²H or D, may afford certain diagnostic and therapeutic advantages resulting from greater metabolic stability by reducing for example defluorination, increased in vivo half-life or reduced dosage requirements, while keeping or improving the original compound efficacy.

Isotopic variations of the compounds of the invention and their precursors can generally be prepared by conventional procedures such as by the illustrative methods or by the preparations described in the Examples and Preparative Examples hereafter using appropriate isotopic variations of suitable reagents, which are commercially available or prepared by known synthetic techniques.

Radionuclides, positron emitters and gamma emitters can be included into the compounds of the present invention and their precursors by methods which are usual in the field of organic synthesis. Typically, they will be introduced by using a correspondingly labelled starting material when the desired compound of the present invention and its precursor is prepared. Illustrative methods of introducing detectable labels are described, for instance, in US 2012/0302755.

The position at which the detectable label is to be attached to the compounds of the present invention and their precursors is not particularly limited. The radionuclides, positron emitters and gamma emitters, for example, can be attached at any position where the corresponding non-emitting atom can also be attached. For instance, ¹⁸F can be attached at any position which is suitable for attaching F. The same applies to the other radionuclides, positron emitters and gamma emitters. Due to the ease of synthesis, preferably R¹ is substituted with ¹⁸F. ³H can be attached at any available position at which H is present. If ²H is employed as a detectable label it can be attached at any available position at which H is present.

In another embodiment, the present invention relates further to a compound of formula (III-F) that is a precursor of the compound of formula (I-F)

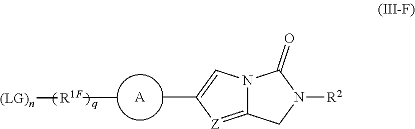

or a stereoisomer, racemic mixture, pharmaceutically acceptable salt, hydrate, or solvate thereof, wherein

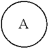

is a 6-membered heteroaryl, which is optionally substituted with at least one halo, haloC₁-C₄alkyl, haloC₁-C₄alkoxy, C₁-C₄alkoxy, or C₁-C₄alkyl;
R^{1F} is a 4- to 6-membered heterocyclyl; or
R^{1F} is C₁-C₄alkoxy, or C₁-C₄alkyl; or
R^{1F} is —NH—C₃-C₆cycloalkyl, C₃-C₆cycloalkyl; and
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
Z is CH or N;
LG is a leaving group;
q is 0 or 1; and
n is at least 1 (e.g., 1, 2 or 3).

In one embodiment, (LG)ₙ-(R^{1F})_{q} is —NH—C₃-C₆cycloalkyl-LG, C₃-C₆cycloalkyl-LG, or heterocyclyl-LG. Preferably (LG)ₙ-R^{1F} is selected from the following:

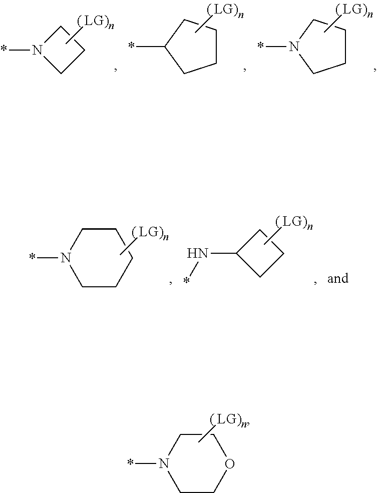

wherein n is at least 1 (e.g., 1, 2, or 3, preferably 1).

In a preferred embodiment, (LG)ₙ-(R^{1F})_{q} is selected from the following:

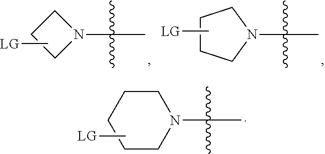

More preferably, (LG)ₙ-R^{1F} is selected from the following:

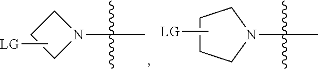

Even more preferably, (LG)ₙ-(R^{1F})_{q} is (LG)ₙ (q is 0) or is:

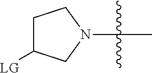

Preferably, the Leaving Group (LG) is halogen, C₁-C₄ alkylsulfonate, C₁-C₄alkyl ammonium, or C₆-C₁₀arylsulfonate, wherein the C₆-C₁₀arylsulfonate can be optionally substituted with —CH₃ or —NO₂. More preferably, the Leaving Group (LG) is nitro, bromo, chloro, iodo, C₁-C₄alkylsulfonate, or C₆-C₁₀arylsulfonate, wherein the C₆-C₁₀arylsulfonate can be optionally substituted with —CH₃ or —NO₂. Even more preferably, the Leaving Group (LG) is mesylate, tosylate or nosylate. Even more preferably, the Leaving Group (LG) is nitro, mesylate, or nosylate. More preferably the Leaving Group (LG) is mesylate or nitro.

In another embodiment, the present invention relates to a compound of formula (III-H), a precursor of the compound of formula (I-H):

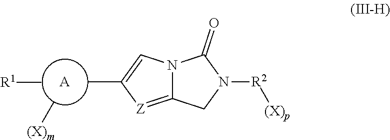

or a stereoisomer, racemic mixture, pharmaceutically acceptable salt, hydrate, or solvate thereof, wherein

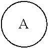

is a 6-membered heteroaryl, which is optionally substituted with at least one halo, haloC₁-C₄alkyl, haloC₁-C₄alkoxy, C₁-C₄alkoxy, or C₁-C₄alkyl;
R¹ is a 4- to 6-membered heterocyclyl which is optionally substituted with at least one halo; or
R¹ is halo, haloC₁-C₄alkyl, haloC₁-C₄alkoxy, C₁-C₄alkoxy, or C₁-C₄alkyl; or
R¹ is —NH—C₃-C₆cycloalkyl or —C₃-C₆cycloalkyl, wherein the C₃-C₆cycloalkyl of —NH—C₃-C₆cycloalkyl or —C₃-C₆cycloalkyl is optionally substituted with at least one halo; and
R² is a 5-membered or 6-membered heteroaryl, optionally substituted with 1 or 2 substituents independently selected from haloC₁-C₄alkyl, haloC₁-C₄alkoxy, C₁-C₄alkoxy, and C₁-C₄alkyl;
Z is CH or N;
X is bromo, chloro or iodo;
m is 0, 1, 2 or 3;
p is 0, 1, 2 or 3; and
with the proviso that the compound of formula (III-H) comprises at least one X (e.g., 1, 2 or 3 X, preferably 1 or 2 X).

In the formula (III-H), halo is preferably F, ¹⁸F or ¹⁹F.

X is attached to the 6-membered heteroaryl of

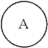

and or the 5-membered or 6-membered heteroaryl of R². If halo is present as a substituent of

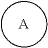

and X is present, the X can be present in addition of halo.

In a preferred embodiment, R² is selected from the following:

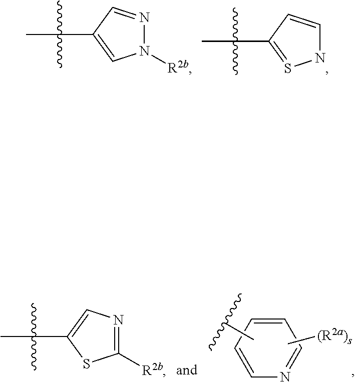

wherein
R^{2a} is independently selected from X, haloC₁-C₄alkyl, haloC₁-C₄alkoxy, C₁-C₄alkoxy, and C₁-C₄alkyl; wherein haloC₁-C₄alkyl, haloC₁-C₄alkoxy, C₁-C₄alkyl, or C₁-C₄alkoxy are optionally substituted with one or more X;
s is 0, 1 or 2 (preferably 0 or 1, more preferably 0); and
R^{2b} is selected from H, X, haloC₁-C₄alkoxy, C₁-C₄alkoxy, and C₁-C₄alkyl.

Preferably, R² is a 5-membered or 6-membered heteroaryl selected from the following:

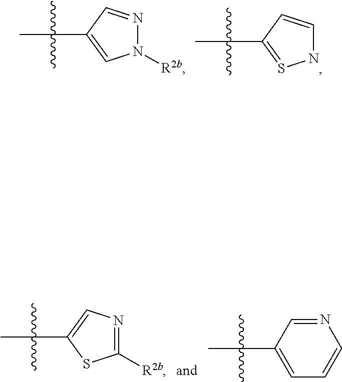

wherein
R^{2b} is selected from H or C₁-C₄alkyl.

More preferably, R² is selected from the following:

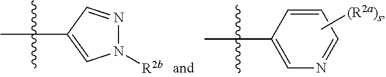

wherein
R^{2a} is X;
R^{2b} is selected from H, X, haloC₁-C₄alkyl, and C₁-C₄alkyl, preferably X;
s is 0, 1 or 2 (preferably 0); and
wherein C₁-C₄alkyl is optionally substituted by one or more X.

In a preferred embodiment, the detectably labelled compound of formula (III-H) comprises one, two or three X. In a preferred embodiment, the detectably labelled compound of formula (III-H) comprises one X. In another preferred embodiment, the detectably labelled compound of formula (III-H) comprises two X. X is selected from bromo, chloro and iodo. In a preferred embodiment X is bromine.

### Methods of Synthesis of Detectably Labelled Compounds

The present invention relates further to a method for preparing a compound of formula (I), or of subformulae thereof (e.g. (I-F), (I-H*), (I-H)), and in particular a compound of formula (III-F) or (III-H).

In one embodiment, the present invention relates to a method for preparing a compound of formula (I-F), by reacting a compound of formula (III-F) with an ¹⁸F-fluorinating agent, so that LG is replaced by ¹⁸F.

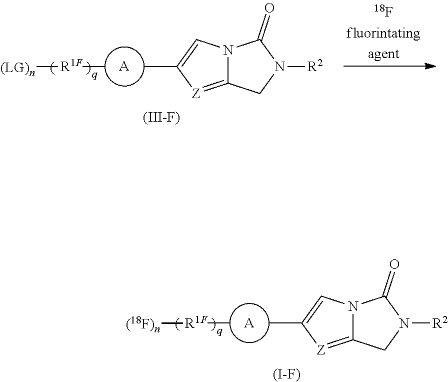

wherein

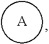

R^{1F}, R², Z, n, q and LG are as defined herein above.

Suitable solvents for the ¹⁸F-fluorination comprise DMF, DMSO, acetonitrile, DMA, or mixtures thereof, preferably acetonitrile or DMSO. Suitable agents for the ¹⁸F-fluorination are selected from K¹⁸F, Rb¹⁸F, Cs¹⁸F, Na¹⁸F, tetra(C₁₋₆alkyl)ammonium salt of ¹⁸F, Kryptofix[222]¹⁸F and tetrabutylammonium [¹⁸F]fluoride.

In one embodiment, the present invention relates to a method of preparing a compound of formula (I-H) by reacting a compound of formula (III-H) with a ³H radiolabeling agent.

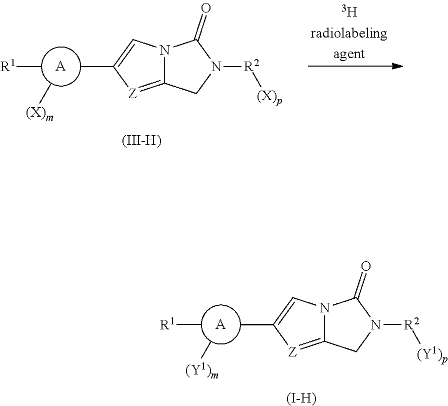

wherein

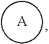

R¹, R², Z, X, Y¹, m, and p are as defined herein above.

In one embodiment, the present invention relates to a method of preparing a compound of formula (I-H*) by reacting a compound of formula (III-H*) with a ³H or D radiolabelling agent.

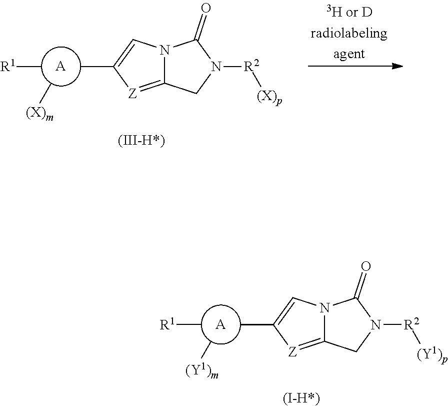

wherein

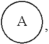

R¹, R², Z, X, m, and p are as defined herein above and wherein Y¹ is D, CD₃, T or CT₃.

The ³H radiolabelling agent can be tritium gas. The method can be conducted in the presence of a catalyst such as palladium on carbon (Pd/C), a solvent such as dimethylformamide (DMF) and a base such as N,N-diisopropylethylamine (DIEA).

In a further embodiment, the compound of formula (III-H*) can be reacted with a ²H radiolabelling agent comprising D (e.g., D₂O, D4-methanol or any other suitable agents), preferably in the presence of a catalyst like Pd/C, so that X is replaced by D (D is deuterium, ²H).

Alternatively, in another embodiment, the present invention relates to a method for preparing a compound of formula (I-H*) or (I-H) by radiolabelling a compound of formula (III-H*) or (III-H), respectively (reacting the compound of formula (III-H*) or (III-H), respectively) with a ³H radiolabelling agent like CT₃ radiolabelling agent, wherein T is ³H, so that X is replaced by CT₃. The CT₃ radiolabelling agent can be ICT₃ (derivative of iodomethane with ³H). The method can be conducted in the presence of a solvent such as dimethylformamide (DMF) and a base such as caesium carbonate or sodium hydride.

### Kits

The precursor compounds of the present invention can also be employed in kits for the preparation of radiopharmaceutical preparations. Due to the radioactive decay, the radiopharmaceuticals are usually prepared immediately before use. The kit typically comprises a precursor of the compound of the present invention, and an agent which reacts with the precursor to introduce a radioactive label into the compound of the present invention. The precursor of the compound of the present invention, can, for example, be a compound having the formula (III-F), (III-H*) or (III-H). The agent can be an agent which introduces a radioactive label such as ¹⁸F, ³H, or D.

In one embodiment, the kit of parts is a test kit for the detection and/or diagnosis of a disease, disorder or abnormality associated with alpha-synuclein aggregates, wherein the test kit comprises at least one precursor of the compound of the present invention (e.g. a compound having the formula (III-F), (III-H*) or (III-H)).

In another embodiment, the kit of parts is a kit for preparing a radiopharmaceutical preparation, wherein the kit comprises a sealed vial containing at least one precursor of the compound of the present invention (e.g. a compound having the formula (III-F), (III-H*) or (III-H)).

In one preferred embodiment, the kit is for use in the imaging of alpha-synuclein aggregates, wherein the imaging is preferably conducted by positron emission tomography, or is for use for in vitro imaging, ex vivo imaging, or in vivo imaging, preferably the use is for in vivo imaging. More preferably, the use is for brain imaging.

### Diagnostic Compositions

The compounds of the present invention are particularly suitable for imaging of alpha-synuclein aggregates. With respect to alpha-synuclein protein, the compounds are particularly suitable for binding to various types of alpha-synuclein aggregates. The imaging can be conducted in mammals, preferably in humans. The imaging is preferably in vitro imaging, ex vivo imaging, or in vivo imaging. More preferably the imaging is in vivo imaging: Even more preferably, the imaging is preferably brain imaging. The imaging can also be eye/retinal imaging. The compounds of the present invention are particularly suitable for use in diagnostics.

The diagnostics can be conducted for mammals, preferably for humans. The tissue of interest on which the diagnostic is conducted can be brain, tissue of the central nervous system, tissue of the eye (such as retinal tissue), tissue of peripheral organs such as the gut or other tissues, or body fluids such as cerebrospinal fluid (CSF) or blood. The tissue is preferably brain tissue.

In one embodiment, the present invention provides a diagnostic composition comprising a compound of the invention, and optionally at least one pharmaceutically acceptable excipient, carrier, diluent and/or adjuvant.

Due to their design and to the binding characteristics, the compounds of the present invention are suitable for use in the diagnosis of diseases, disorders and abnormalities associated with alpha-synuclein aggregates. In another embodiment, the diagnostic composition which comprises a compound of the present invention is also suitable for use in the diagnosis of diseases, disorders and abnormalities associated with alpha-synuclein aggregates.

In yet another embodiment, the compound of the present invention, or the diagnostic composition comprising a compound of the invention, is suitable for use in imaging, such as in vitro imaging, ex vivo imaging, or in vivo imaging, preferably the use is for in vivo imaging, more preferably the use is for brain imaging. In particular, the use is in humans.

In another embodiment, the compounds of the present invention or the diagnostic composition are particularly suitable for use in positron emission tomography imaging of alpha-synuclein aggregates.

Diseases involving alpha-synuclein aggregates are generally listed as synucleinopathies (or α-synucleinopathies). The compounds of the present invention are suitable for use in the diagnosis of diseases, disorders or abnormalities associated with alpha-synuclein aggregates including, but not limited to, Lewy bodies and/or Lewy neurites or a predisposition therefor, wherein the diseases, disorders or abnormalities are selected from (including, but not limited to) Parkinson's disease (sporadic, familial with alpha-synuclein mutations, familial with mutations other than alpha-synuclein, pure autonomic failure and Lewy body dysphagia), SNCA duplication carrier, dementia with Lewy bodies (“pure” Lewy body dementia), Alzheimer's disease, sporadic Alzheimer's disease, familial Alzheimer's disease with APP mutations, familial Alzheimer's disease with PS-1, PS-2 or other mutations, familial British dementia, Lewy body variant of Alzheimer's disease and normal aging in Down syndrome). The compounds of the present invention are suitable for use in the diagnosis of diseases, disorders or abnormalities associated with alpha-synuclein aggregates including, but not limited to neuronal and glial aggregates of alpha synuclein including multiple system atrophy (MSA) (Shy-Drager syndrome, striatonigral degeneration and olivopontocerebellar atrophy). Other diseases that may have alpha-synuclein-immunoreactive lesions include traumatic brain injury, chronic traumatic encephalopathy, tauopathies (Pick's disease, frontotemporal dementia, progressive supranuclear palsy, corticobasal degeneration and Niemann-Pick type C1 disease), motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, ataxia telangiectatica, Meige's syndrome, subacute sclerosing panencephalitis, Gaucher disease as well as other lysosomal storage disorders (including Kufor-Rakeb syndrome and Sanfilippo syndrome) and rapid eye movement (REM) sleep behavior disorder (Jellinger, Mov Disord 2003, 18 Suppl. 6, S2-12; Galvin et al. JAMA Neurology 2001, 58 (2), 186-190; Kovari et al., Acta Neuropathol. 2007, 114(3), 295-8; Saito et al., J Neuropathol Exp Neurol. 2004, 63(4), 323-328; McKee et al., Brain, 2013, 136(Pt 1), 43-64; Puschmann et al., Parkinsonism Relat Disord 2012, 18S1, S24-S27; Usenovic et al., J Neurosci. 2012, 32(12), 4240-4246; Winder-Rhodes et al., Mov Disord. 2012, 27(2), 312-315; Ferman et al., J Int Neuropsychol Soc. 2002, 8(7), 907-914). Preferably, the compounds of the present invention are suitable for use in the diagnosis of Parkinson's disease, multiple system atrophy, dementia with Lewy bodies, Parkinson's disease dementia, SNCA duplication carrier, or Alzheimer's disease, more preferably Parkinson's disease (PD) or multiple system atrophy (MSA).

In the present invention, the disease, disorder or abnormality can be selected from Parkinson's disease (including sporadic, familial with alpha-synuclein mutations, familial with mutations other than alpha-synuclein, pure autonomic failure or Lewy body dysphagia), SNCA duplication carrier, Lewy Body dementia (LBD), dementia with Lewy bodies (DLB) (including “pure” Lewy body dementia), Parkinson's disease dementia (PDD), diffuse Lewy body disease (DLBD), Alzheimer's disease, sporadic Alzheimer's disease, familial Alzheimer's disease with APP mutations, familial Alzheimer's disease with PS-1, PS-2 or other mutations, familial British dementia, Lewy body variant of Alzheimer's disease, Down syndrome, multiple system atrophy (MSA) (including Shy-Drager syndrome, striatonigral degeneration or olivopontocerebellar atrophy), traumatic brain injury, chronic traumatic encephalopathy, dementia puglistica, tauopathies (including Pick's disease, frontotemporal dementia, progressive supranuclear palsy, corticobasal degeneration, Niemann-Pick type C1 disease, frontotemporal dementia with Parkinsonism linked to chromosome 17), Creutzfeldt-Jakob disease, Huntington's disease, motor neuron disease, amyotrophic lateral sclerosis (including sporadic, familial or ALS-dementia complex of Guam), neuroaxonal dystrophy, neurodegeneration with brain iron accumulation type 1 (including Hallervorden-Spatz syndrome), prion diseases, ataxia telangiectatica, Meige's syndrome, subacute sclerosing panencephalitis, Gerstmann-Straussler-Scheinker disease, inclusion-body myositis, Gaucher disease, Krabbe disease as well as other lysosomal storage disorders (including Kufor-Rakeb syndrome and Sanfilippo syndrome) and rapid eye movement (REM) sleep behavior disorder.

In the methods of diagnosing a disease, disorder or abnormality associated with alpha-synuclein aggregates (e.g. Parkinson's disease) or MSA, or a predisposition therefor in a subject, the method comprises the steps of:
(a) administering to the subject a diagnostically effective amount of a compound of the present invention, or a diagnostic composition which comprises a compound of the present invention;
(b) allowing the compound of the present invention to distribute into the tissue of interest (such as brain tissue, tissue of the central nervous system (CNS), tissue of the eye, tissue of peripheral organs or other tissues), or body fluid (such as cerebrospinal fluid (CSF) or blood); and
(c) imaging the tissue of interest or body fluid.

If the amount of the compound bound to the alpha-synuclein aggregates, is increased compared to a normal control level the subject is suffering from or is at risk of developing a disease, disorder or abnormality associated with alpha-synuclein aggregates.

The compounds of the present invention can be used for imaging of alpha-synuclein aggregates in any sample or a specific body part or body area of a patient which is suspected to contain alpha-synuclein aggregates. The compounds are able to pass the blood-brain barrier. Consequently, they are particularly suitable for imaging of alpha-synuclein aggregates in the brain, tissue of the central nervous system (CNS), tissue of the eye (such as retinal tissue), tissue of peripheral organs such as the gut or other tissues, or body fluids such as cerebrospinal fluid (CSF) or blood.

In diagnostic applications, the compounds of the present invention are preferably administered in the form of a diagnostic composition comprising the compound of the invention. A “diagnostic composition” is defined in the present invention as a composition comprising one or more compounds of the present invention in a form suitable for administration to a patient, e.g., a mammal such as a human, and which is suitable for use in the diagnosis of the specific disease, disorder or abnormality at issue. Preferably a diagnostic composition further comprises a pharmaceutically acceptable excipient, carrier, diluent or adjuvant. Administration is preferably carried out as defined below. More preferably by injection of the composition as an aqueous solution. Such a composition may optionally contain further ingredients such as buffers; pharmaceutically acceptable solubilizers (e.g., cyclodextrins or surfactants such as Pluronic, Tween or phospholipids); and pharmaceutically acceptable stabilisers or antioxidants (such as ascorbic acid, gentisic acid or para-aminobenzoic acid). The dose of the compound of the present invention will vary depending on the exact compound to be administered, the weight of the patient, and other variables as would be apparent to a physician skilled in the art.

While it is possible for the compounds of the present invention to be administered alone, it is preferable to formulate them into a diagnostic composition in accordance with standard pharmaceutical practice. Thus, the invention also provides a diagnostic composition which comprises a diagnostically effective amount of a compound of the present invention in admixture with, optionally, at least one pharmaceutically acceptable excipient, carrier, diluent or adjuvant.

Pharmaceutically acceptable excipients are well known in the pharmaceutical art, and are described, for example, in Remington's Pharmaceutical Sciences, 15^{th} Ed., Mack Publishing Co., New Jersey (1975). The pharmaceutical excipient can be selected with regard to the intended route of administration and standard pharmaceutical practice. The excipient must be acceptable in the sense of being not deleterious to the recipient thereof.

Pharmaceutically useful excipients, carriers, adjuvants and diluents that may be used in the formulation of the diagnostic composition of the present invention may comprise, for example, solvents such as monohydric alcohols such as ethanol, isopropanol and polyhydric alcohols such as glycols and edible oils such as soybean oil, coconut oil, olive oil, safflower oil, cottonseed oil, oily esters such as ethyl oleate, isopropyl myristate, binders, adjuvants, solubilizers, thickening agents, stabilizers, disintegrants, glidants, lubricating agents, buffering agents, emulsifiers, wetting agents, suspending agents, sweetening agents, colorants, flavors, coating agents, preservatives, antioxidants, processing agents, drug delivery modifiers and enhancers such as calcium phosphate, magnesium stearate, talc, monosaccharides, disaccharides, starch, gelatin, cellulose, methylcellulose, sodium carboxymethyl cellulose, dextrose, hydroxypropyl-β-cyclodextrin, polyvinylpyrrolidone, low melting waxes, and ion exchange resins.

The routes for administration (delivery) of the compounds of the invention include, but are not limited to, one or more of: intravenous, gastrointestinal, intraspinal, intraperitoneal, intramuscular, oral (e. g. as a tablet, capsule, or as an ingestible solution), topical, mucosal (e. g. as a nasal spray or aerosol for inhalation), nasal, parenteral (e. g. by an injectable form), intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual. Preferably, the route of administration (delivery) of the compounds of the invention is intravenous.

For example, the compounds can be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include starch, a cellulose, milk sugar (lactose) or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Preferably, in diagnostic applications, the compounds of the present invention are administered parenterally. If the compounds of the present invention are administered parenterally, then examples of such administration include one or more of: intravenously, intraarterially, intraperitoneally, intrathecally, intraventricularly, intraurethrally, intrasternally, intracranially, intramuscularly or subcutaneously administering the compounds; and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

As indicated, the compounds of the present invention can be administered intranasally or by inhalation and are conveniently delivered in the form of a dry powder inhaler or an aerosol spray presentation from a pressurized container, pump, spray or nebulizer with the use of a suitable propellant, e.g. dichlorodifluoromethane, trichlorofluoromethane, dichlorotetrafluoroethane, a hydrofluoroalkane such as 1,1,1,2-tetrafluoroethane (HFA134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e. g. using a mixture of ethanol and the propellant as the solvent, which may additionally contain a lubricant, e. g. sorbitan trioleate. Capsules and cartridges (made, for example, from gelatin) for use in an inhaler or insufflator may be formulated to contain a powder mix of the compound and a suitable powder base such as lactose or starch.

Alternatively, the compounds of the present invention can be administered in the form of a suppository or pessary, or it may be applied topically in the form of a gel, hydrogel, lotion, solution, cream, ointment or dusting powder. The compounds of the present invention may also be dermally or transdermally administered, for example, by the use of a skin patch.

They may also be administered by the pulmonary or rectal routes. They may also be administered by the ocular route. For ophthalmic use, the compounds can be formulated as micronized suspensions in isotonic, pH was adjusted, sterile saline, or, preferably, as solutions in isotonic, pH was adjusted, sterile saline, optionally in combination with a preservative such as a benzylalkonium chloride. Alternatively, they may be formulated in an ointment such as petrolatum.

For application topically to the skin, the compounds of the present invention can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing diagnosis.

The diagnostic compositions of the invention can be produced in a manner known per se to the skilled person as described, for example, in Remington's Pharmaceutical Sciences, 15^{th} Ed., Mack Publishing Co., New Jersey (1975).

The compounds of the present invention are useful as an in vitro analytical reference or an in vitro screening tool. They are also useful in in vivo diagnostic methods.

The compounds according to the present invention can also be provided in the form of a mixture, a pharmaceutical composition, or a combination, comprising a compound according to the present invention and at least one compound selected from an imaging agent different from the compound according to the invention, a pharmaceutically acceptable excipient, carrier, diluent or adjuvant. The imaging agent different from the compound according to the invention is preferably present in a diagnostically effective amount. More preferably the imaging agent different from the compound according to the invention is an Abeta or Tau imaging agent.

### Methods of Using the Invention

In one embodiment, the invention provides a method of diagnosing a disease, disorder or abnormality associated with alpha-synuclein aggregates, in a subject, the method comprising the steps:
(a) Administering a compound of the invention, or a diagnostic composition which comprises a compound of the invention to the subject;
(b) Allowing said compound to bind to the alpha-synuclein aggregates; and
(c) Detecting the compound bound to the alpha-synuclein aggregates.

Optionally, said method may further comprise the step of:
(d) Generating an image representative of the location and/or amount of the compound bound to the alpha-synuclein aggregates.

In another embodiment, the invention provides a method of positron emission tomography (PET) imaging of alpha-synuclein aggregates in a tissue of a subject, the method comprising the steps:
(a) Administering a compound of the invention, or a diagnostic composition which comprises a compound of the invention to the subject;
(b) Allowing the compound to bind to the alpha-synuclein aggregates; and
(c) Detecting the compound bound to the alpha-synuclein aggregates by collecting a positron emission tomography (PET) image of the tissue of the subject;

In another embodiment, the invention relates to a method for the detection and optionally quantification (e.g., an in vivo or in vitro method) of alpha-synuclein aggregates in a tissue of a subject, the method comprising the steps:
(a) Bringing a sample or a specific body part or body area suspected to contain alpha-synuclein aggregates into contact with a compound of the invention, or a diagnostic composition which comprises a compound of the invention;
(b) Allowing the compound to bind to the alpha-synuclein aggregates;
(c) Detecting the compound bound to the alpha-synuclein aggregates; and
(d) Optionally quantifying the amount of the compound bound to the alpha-synuclein aggregates.

In an embodiment, the present invention refers to a method of collecting data for the diagnosis of a disease, disorder or abnormality associated with alpha-synuclein aggregates, the method comprising the steps:
(a) Bringing a sample or a specific body part or body area suspected to contain alpha-synuclein aggregates into contact with a compound according to the present invention, or a diagnostic composition which comprises a compound according to the present invention;
(b) Allowing the compound to bind to the alpha-synuclein aggregates;
(c) Detecting the compound bound to the alpha-synuclein aggregates; and
(d) Optionally correlating the presence or absence of the compound bound to the alpha-synuclein aggregates with the presence or absence of the alpha-synuclein aggregates in the sample or specific body part or body area.

If the amount of the compound bound to the alpha-synuclein aggregates is higher than a normal control value it can be assumed that the patient is suffering from a disease, disorder or abnormality associated with alpha-synuclein aggregates.

Yet another embodiment of the present invention refers to a method of collecting data for determining a predisposition to a disease, disorder or abnormality associated with alpha-synuclein aggregates, the method comprising the steps:
(a) Bringing a sample or a specific body part or body area suspected to contain alpha-synuclein aggregates into contact with a compound according to the present invention, or a diagnostic composition which comprises a compound according to the present invention;
(b) Allowing the compound to bind to the alpha-synuclein aggregates;
(c) Detecting the compound bound to the alpha-synuclein aggregates; and
(d) Optionally correlating the presence or absence of the compound bound to the alpha-synuclein aggregates with the presence or absence of the alpha-synuclein aggregates in the sample or specific body part or body area.

If the amount of the compound bound to the alpha-synuclein aggregates is higher than a normal control value of a healthy/reference subject this indicates that the patient is suffering from or is at risk of developing a disease, disorder or abnormality associated with alpha-synuclein aggregates. In particular, if the amount of the compound bound to the alpha-synuclein aggregates is higher than what expected in a person showing no clinical evidence of a disease, disorder or abnormality associated with alpha-synuclein aggregates, it can be assumed that the patient has a disposition to a disease, disorder or abnormality associated with alpha-synuclein aggregates.

In a further aspect, the present invention relates to a method of collecting data for prognosing a disease, disorder or abnormality associated with alpha-synuclein aggregates, wherein the method comprises the steps:
(a) Bringing a sample, a specific body part or body area suspected to contain alpha-synuclein aggregates into contact with a compound according to the present invention, or a diagnostic composition which comprises a compound according to the present invention;
(b) Allowing the compound to bind to the alpha-synuclein aggregates;
(c) Detecting the compound bound to the alpha-synuclein aggregates;
(d) Optionally correlating the presence or absence of the compound bound to the alpha-synuclein aggregates with the presence or absence of the alpha-synuclein aggregates in the sample or specific body part or body area; and
(e) Optionally repeating steps (a) to (c) and, if present, optional step (d) at least one time.

The progression of a disease, disorder or abnormality and/or the prospect (e.g., the probability, duration, and/or extent) of recovery can be estimated by a medical practitioner based on the presence or absence of the compound bound to the alpha-synuclein aggregates, the amount of the compound bound to the alpha-synuclein aggregates or the like. If desired, steps (a) to (c) and, if present, optional step (d) can be repeated over time to monitor the progression of the disease, disorder or abnormality and to thus allow a more reliable estimate.

A further aspect is directed to a method of collecting data for monitoring the progression (or evolution) of a disease, disorder or abnormality associated with alpha-synuclein aggregates in a patient, the method comprising the steps:
(a) Bringing a sample, a specific body part or body area suspected to contain alpha-synuclein aggregates into contact with the compound according to the present invention, or a diagnostic composition which comprises a compound according to the present invention;
(b) Allowing the compound to bind to the alpha-synuclein aggregates;
(c) Detecting the compound bound to the alpha-synuclein aggregates;
(d) Optionally correlating the presence or absence of the compound bound to the alpha-synuclein aggregates with the presence or absence of the alpha-synuclein aggregates in the sample or specific body part or body area; and
(e) Optionally repeating steps (a) to (c) and, if present, optional step (d) at least one time.

In the method for monitoring the progression the amount of the compound bound to the alpha-synuclein aggregates can be optionally compared at various points of time during the treatment, for instance, before and after onset of the treatment or at various points of time after the onset of the treatment.

Typically, the patient is or has been undergoing treatment of the disease, disorder or abnormality associated with alpha-synuclein aggregates or is/has been undergoing treatment of the synucleinopathy. In particular, the treatment can involve administration of a medicament which is suitable for treating the disease, disorder or abnormality associated with alpha-synuclein aggregates.

In another embodiment, the invention relates to a method of collecting data for predicting responsiveness of a patient suffering from a disease, disorder or abnormality associated with alpha-synuclein aggregates to a treatment with a medicament, the method comprising the steps of
(a) Bringing a sample, a specific body part or body area suspected to contain alpha-synuclein aggregates into contact with a compound of the invention, or a diagnostic composition which comprises a compound of the invention;
(b) Allowing the compound to bind to the alpha-synuclein aggregates;
(c) Detecting the compound bound to the alpha-synuclein aggregates;
(d) Optionally correlating the presence or absence of the compound bound to the alpha-synuclein aggregates with the presence or absence of the alpha-synuclein aggregates in the sample or specific body part or body area; and
(e) Optionally repeating steps (a) to (c) and, if present, optional step (d) at least one time.

In the method for predicting the responsiveness, the method can further comprises steps (i) to (vi) before step (a):
(i) bringing a sample or specific body part or body area suspected to contain alpha-synuclein aggregates into contact with the compound of the present invention, which compound specifically binds to the alpha-synuclein aggregates;
(ii) allowing the compound to bind to the alpha-synuclein aggregates;
(iii) detecting the formation of the compound bound to the alpha-synuclein aggregates;
(iv) optionally correlating the presence or absence of the compound bound to the alpha-synuclein aggregates with the presence or absence of alpha-synuclein aggregates in the sample or specific body part or body area;
(v) optionally comparing the amount of the compound bound to the alpha-synuclein aggregates to a normal control value; and
(vi) treating the patient with the medicament.

Optionally the method can further comprise step (A) after step (d) or step (e):
(A) comparing the amount of the compound bound to the alpha-synuclein aggregates determined in step (iv) to the amount of the compound bound to the alpha-synuclein aggregates determined in step (d).

In the method for predicting responsiveness the amount of the compound bound to the alpha-synuclein aggregates can be optionally compared at various points of time during the treatment, for instance, before and after onset of the treatment or at various points of time after the onset of the treatment. A change, especially a decrease, in the amount of the compound bound to the alpha-synuclein aggregates may indicate that the patient has a high potential of being responsive to the respective treatment.

If the amount of the compound bound to the alpha-synuclein aggregates decreases over time, it can be assumed that the patient is responsive to the treatment. If the amount of the compound bound to the alpha-synuclein aggregates is essentially constant or increases overtime, it can be assumed that the patient is non-responsive to the treatment.

Alternatively, the responsiveness can be estimated by determining the amount of the compound bound to the alpha-synuclein aggregates. The amount of the compound bound to the alpha-synuclein aggregates can be compared to a control value such as a normal control value, a preclinical control value or a clinical control value. Alternatively, the control value may refer to the control value of subjects known to be responsive to a certain therapy, or the control value may refer to the control value of subjects known to be non-responsive to a certain therapy. The outcome with respect to responsiveness can either be “responsive” to a certain therapy, “non-responsive” to a certain therapy or “response undetermined” to a certain therapy. Response to the therapy may be different for the respective patients.

Optionally, the diagnostic composition can be used before, during and after, surgical procedures (e.g. deep brain stimulation (DBS)) and non-invasive brain stimulation (such as repetitive transcranial magnetic stimulation (rTMS)), for visualizing alpha-synuclein aggregates before, during and after such procedures. Surgical techniques, including DBS, improve advanced symptoms of PD on top of the best currently used medical therapy. During the past 2 decades, rTMS has been closely examined as a possible treatment for PD (Ying-hui Chou et al. JAMA Neurol. 2015 Apr. 1; 72(4): 432-440).

In any of the above methods, the step of optionally correlating the presence or absence of the compound bound to the alpha-synuclein aggregates with the presence or absence of the alpha-synuclein aggregates in the sample or specific body part or body area; comprises
determining the amount of the compound bound to the alpha-synuclein aggregates;
correlating the amount of the compound bound to the alpha-synuclein aggregates with the amount of the alpha-synuclein aggregates in the sample or specific body part or body area; and
optionally comparing the amount of the compound bound with the alpha-synuclein aggregates in the sample or specific body part or body area to a normal control value in a healthy control subject.

The control value can be, e.g., a normal control value, a preclinical control value and/or a clinical control value.

A “healthy control subject” or “healthy volunteer (HV) subject” is a person showing no clinical evidence of a disease, disorder or abnormality associated with alpha-synuclein aggregates.

In an embodiment of any of the above methods the alpha-synuclein aggregates include, but are not limited to, Lewy bodies and/or Lewy neurites.

If in any of the above summarized methods the amount of the compound bound with the alpha-synuclein aggregates is higher than the normal control value, then it can be expected that the patient is suffering from or is likely to from a disease, disorder or abnormality associated with alpha-synuclein aggregates or from a synucleinopathy.

A sample or a specific body part or body area suspected to contain alpha-synuclein aggregates is brought into contact with a compound of the present invention.

Any of the compounds of the present invention can be used in the above summarized methods. Preferably detectably labelled compounds of the present invention are employed in the above summarized methods.

The specific body part or body area is preferably of a mammal, more preferably of a human, including the full body or partial body area or body part of the patient suspected to contain alpha-synuclein aggregates. The specific body part or body area can be brain, the central nervous system, eye or a peripheral organ such as the gut, preferably brain.

The tissue can be brain tissue, tissue of the central nervous system (CNS), tissue of the eye (such as retinal tissue), tissue of peripheral organs such as the gut or other tissues, or body fluids such as cerebrospinal fluid (CSF) or blood. The tissue is preferably brain tissue. Preferably, the sample is an in vitro sample from a patient.

In the above methods, the compound of the present invention can be brought into contact with the sample or the specific body part or body area suspected to contain the alpha-synuclein aggregates by any suitable method.

In in vitro methods the compound of the present invention and a liquid sample can be simply mixed.

In an in vivo method, the specific body part or body area can be brought into contact with a compound of the invention by administering an effective amount of a compound of the invention to the patient.

The effective amount of a compound of the invention is an amount which is suitable for allowing the presence or absence of alpha-synuclein aggregates in the sample, specific body part or body area to be determined using the chosen analytical technique. The amount is not particularly limited and will depend on the compound of the formula (I), the type of detectable label, the sensitivity of the respective analytical method and the respective device. The amount can be chosen appropriately by a skilled person.

The compound is then allowed to bind to the alpha-synuclein aggregates, including but not limited to, Lewy bodies and/or Lewy neurites. The step of allowing the compound to bind to the alpha-synuclein aggregates includes allowing sufficient time for the compound of the invention to bind to the alpha-synuclein aggregates. The amount of time required for binding will depend on the type of test (e.g., in vitro or in vivo) and can be determined by a person skilled in the field by routine experiments. In an in vivo method, the amount of time will depend on the time which is required for the compound to reach the specific body part or body area suspected to contain alpha-synuclein aggregates. The amount of time should not be too extended to avoid washout and/or metabolism of the compound of the invention.

The compound which has bound to the alpha-synuclein aggregates can be subsequently detected by any appropriate method. The method of detecting the compound bound to the alpha-synuclein aggregates is not particularly limited and depends, among others, on the detectable label, the type of sample, specific body part or body area and whether the method is an in vitro or in vivo method. Examples of possible methods include, but are not limited to, a fluorescence imaging technique or a nuclear imaging technique such as positron emission tomography (PET), single photon emission computed tomography (SPECT), magnetic resonance imaging (MRI), and contrast-enhanced magnetic resonance imaging (MRI). These have been described and enable visualization of alpha-synuclein biomarkers. The fluorescence imaging technique and/or nuclear imaging technique can be employed for monitoring and/or visualizing the distribution of the detectably labelled compound within the sample or a specific body part or body area. The imaging system provides an image of bound detectable label such as radioisotopes, in particular positron emitters or gamma emitters, as present in the tested sample, the tested specific body part or the tested body area. Preferably, the compound bound to the alpha-synuclein aggregates is detected by an imaging apparatus such as PET or SPECT scanner, more preferably PET.

The amount of the compound bound to the alpha-synuclein aggregates can be determined by visual or quantitative analysis, for example, using PET scan images.

A compound according to the present invention or its precursor can also be incorporated into a test kit for detecting alpha-synuclein protein aggregates. The test kit typically comprises a container holding one or more compounds according to the present invention or its precursor(s) and instructions for using the compound for the purpose of binding to alpha-synuclein aggregates and detecting the formation of the compound bound to the alpha-synuclein aggregates such that presence or absence of the compound bound to the alpha-synuclein aggregates correlates with the presence or absence of the alpha-synuclein aggregates.

The term “test kit” refers in general to any diagnostic kit known in the art. More specifically, the latter term refers to a diagnostic kit as described in Zrein et al., Clin. Diagn. Lab. Immunol., 1998, 5, 45-49.

The dose of the detectably labelled compounds of the present invention, preferably compounds of formula (I-F) labelled with ¹⁸F or compounds of formula (I-H*) or (I-H) labelled with ³H, will vary depending on the exact compound to be administered, the weight of the patient, size and type of the sample, and other variables as would be apparent to a physician skilled in the art. Generally, the dose could preferably lie in the range 0.001 μg/kg to 10 μg/kg, preferably 0.01 μg/kg to 1.0 μg/kg. The radioactive dose can be, e.g., 100 to 600 MBq, more preferably 150 to 450 MBq.

### Methods of Synthesizing the Compounds of the Invention

The compounds of the present invention may be prepared in accordance with the definition of compound of formula (I) by the routes described in the following Schemes or the Examples. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. In the following general methods, R¹, R², \textcircled{A}, Z, Y¹, LG, Hal, m and n are as previously defined in the above embodiments or limited to designations in the Schemes. Unless otherwise stated, starting materials are either commercially available or are prepared by known methods.

### General Synthetic Schemes for the Preparation of Compounds of this Invention

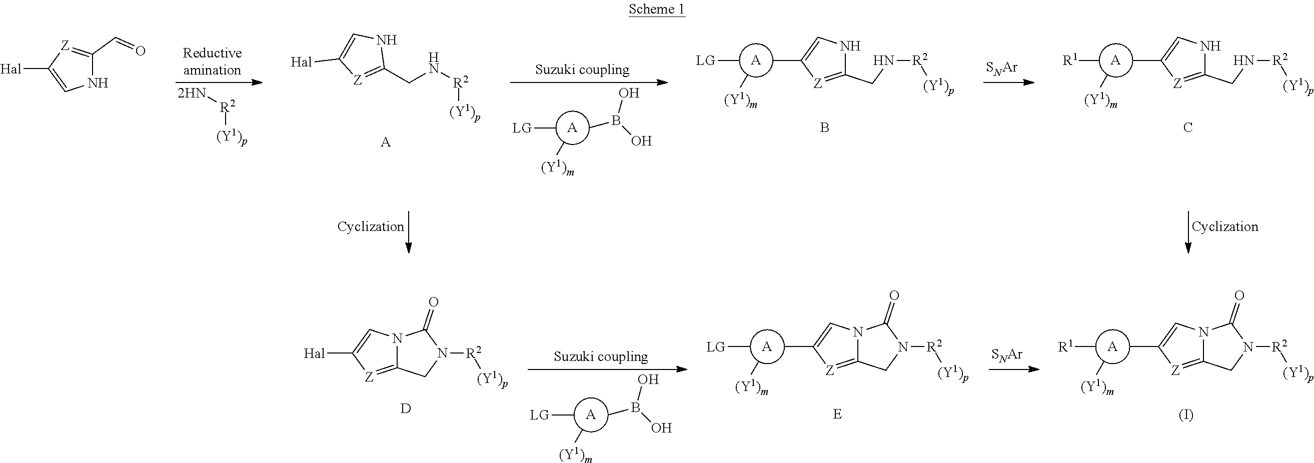

Commercially available or custom-made aldehyde derivatives can be reacted with a suitable amine via reductive amination to obtain intermediate A after purification. The halogen atom is then replaced by a heteroaryl boronic acid derivative containing a leaving group (LG) via Suzuki coupling to obtain intermediate B after purification. The LG is then substituted with a suitable amine derivative via aromatic nucleophilic substitution to obtain intermediate C after purification. Intermediate C can be ring cyclized using 1,1′-carbonyldiimidazole (CDI) in an appropriate solvent to afford compounds of formula (I). Alternatively, commercially available or custom-made aldehyde can be ring cyclized using 1,1′-carbonyldiimidazole (CDI) in an appropriate solvent to afford intermediate D. The halogen atom is then replaced by a heteroaryl boronic acid derivative containing a leaving group (LG) via Suzuki coupling to obtain intermediate E after purification. The LG is then substituted with a suitable amine derivative via aromatic nucleophilic substitution to obtain compounds of formula (I).

### General Synthesis of ¹⁸F-Labelled Compounds of the Present Invention

Compounds having the formula (I) which are labelled by ¹⁸F can be prepared by reacting a precursor compound (III-F), as described below, with an ¹⁸F-fluorinating agent, so that the LG comprised in the precursor compound is replaced by ¹⁸F.

The reagents, solvents and conditions which can be used for the ¹⁸F-fluorination are well-known to a skilled person in the field (L. Cai, S. Lu, V. Pike, Eur. J. Org. Chem 2008, 2853-2873; J. Fluorine Chem., 27 (1985):177-191; Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). Preferably, the solvents used in the ¹⁸F-fluorination are DMF, DMSO, acetonitrile, DMA, or mixtures thereof, preferably the solvent is acetonitrile or DMSO.

Any suitable ¹⁸F-fluorinating agent can be employed. Typical examples include H¹⁸F, alkali or alkaline earth ¹⁸F-fluorides (e.g., K¹⁸F, Rb¹⁸F, Cs¹⁸F, and Na¹⁸F). Optionally, the ¹⁸F-fluorination agent can be used in combination with a chelating agent such as a cryptand (e.g.: 4,7,13,16,21,24-hexaoxa-1,10-diazabicyclo[8.8.8]-hexacosane—Kryptofix®) or a crown ether (e.g.: 18-crown-6). Alternatively, the ¹⁸F-fluorinating agent can be a tetraalkylammonium salt of ¹⁸F or a tetraalkylphosphonium salt of ¹⁸F; e.g., tetra(C₁₋₆ alkyl)ammonium salt of ¹⁸F or a tetra(C₁₋₆ alkyl)phosphonium salt of ¹⁸F. Preferably, the ¹⁸F-fluorination agent is K¹⁸F, H¹⁸F, Cs¹⁸F, Na¹⁸F, tetra(C₁₋₆ alkyl) ammonium salt of ¹⁸F, Kryptofix[222]¹⁸F or tetrabutylammonium [¹⁸F]fluoride.

Although the reaction is shown above with respect to ¹⁸F as a radioactive label, other radioactive labels can be introduced following similar procedures.

The invention is illustrated by the following examples which, however, should not be construed as limiting.

### EXAMPLES

Compounds of the present disclosure may be prepared by methods known in the art of organic synthesis. In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (2014) Protective Groups in Organic Synthesis, 5th edition, John Wiley & Sons). These groups are removed at a convenient stage of the compound synthesis using methods that are readily apparent to those skilled in the art.

Unless otherwise noted, all reagents and solvents were obtained from commercial sources and used without further purification.

The chemical names were generated using ChemBioDraw Ultra v20 from CambridgeSoft.

Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials was confirmed by standard analytical methods, e.g., microanalysis and spectroscopic characteristics, e.g., MS, IR, NMR.

### Abbreviations

Abbreviations used are those conventional in the art.

| | |
|---|---|
| CDI | carbonyldiimidazole |
| DCM | dichloromethane |
| DMSO | dimethylsulfoxide |
| DTT | dithiothreitol |
| EGTA | ethylene glycol-bis(β-aminoethyl ether)-N,N,N′,N′- |
| | tetraacetic acid |
| HPLC | High Performance Liquid Chromatography |
| LCMS | Liquid Chromatography Mass Spectrometry |
| LG | leaving group |
| MS | mass spectra |
| NMR | nuclear magnetic resonance- |
| Pg | protecting group |
| S_{N}Ar | nucleophilic aromatic substitution |
| THF | tetrahydrofuran |
| TMS | tetramethylsilane |
| RT | room temperature |

### Analytical Details, Preparative and Analytical Methods

NMR measurements were performed on a DRX-400 MHz NMR spectrometer, on a Bruker AV-400 MHz NMR spectrometer or Spinsolve 80 MHz NMR spectrometer in deuterated solvents, using or not tetramethylsilane (TMS) as an internal standard. Chemical shifts (δ) are reported in ppm downfield from TMS, spectra splitting patterns are designated as singlet (s), doublet (d), triplet (t), quartet (q), quintet (quint), septet (sept), multiplet, unresolved or overlapping signals (m), or broad signal (br). Deuterated solvents are given in parentheses and have chemical shifts of dimethyl sulfoxide (δ 2.50 ppm), methanol (δ 3.31 ppm), chloroform (δ 7.26 ppm), or other solvent as indicated in NMR spectral data.

Mass spectra (MS) were recorded on an Advion CMS mass spectrometer or an UPLC H-Class Plus with Photodiode Array detector and Qda Mass spectrometer from Waters.

Column chromatography was performed using silica gel (Fluka: Silica gel 60, 0.063-0.2 mm) and suitable solvents as indicated in the specific examples.

Flash Column Chromatography System: flash purification was conducted with a Biotage Isolera One flash purification system using HP-Sil or KP-NH SNAP cartridges (Biotage) and the solvent gradient indicated in the specific examples.

Thin layer chromatography (TLC) was carried out on silica gel plates with UV detection.

### Example 1: (R)-6-(6-(3-fluoropyrrolidin-1-yl)pyridin-3-yl)-2-(pyridin-3-yl)-1H-pyrrolo[1,2-c]imidazol-3(2H)-one Hydrochloride

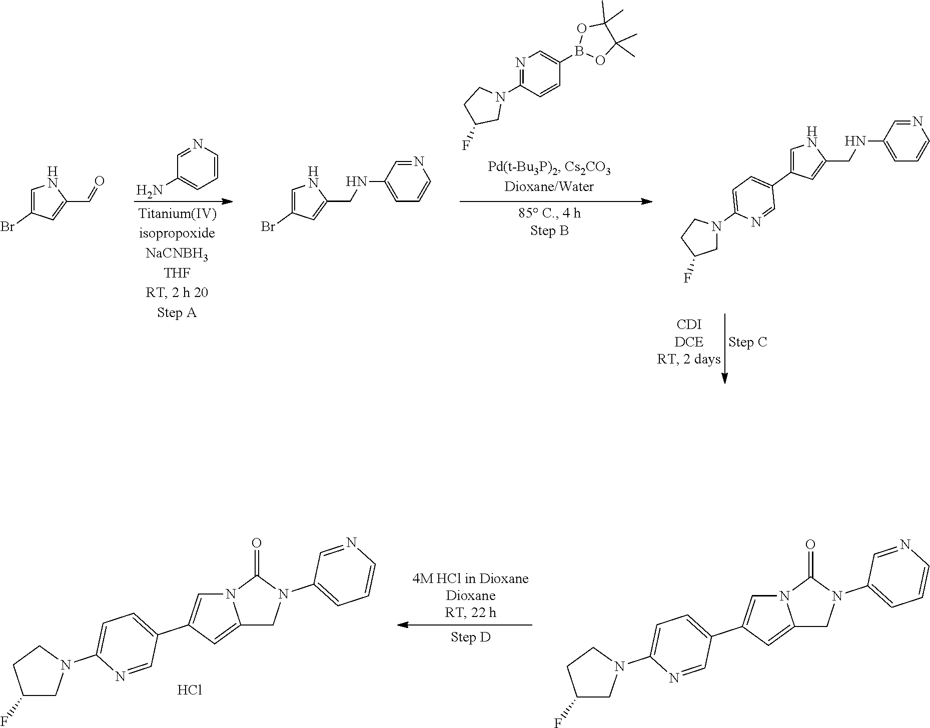

### Step A:

To a solution of 4-bromo-1H-pyrrole-2-carbaldehyde (1.5 g, 8.62 mmol) in tetrahydrofuran (60 mL) was added titanium (IV) isopropoxide (2.53 mL, 8.62 mmol), followed by 3-aminopyridine (2.434 g, 25.9 mmol). The reaction mixture was stirred for 40 min at room temperature, followed by the addition of sodium cyanoborohydride (0.813 g, 12.93 mmol). The reaction mixture was stirred for 40 min but the starting material was still present. Thus, another batch of titanium (IV) isopropoxide (2.53 mL, 8.62 mmol) was added and the reaction mixture was stirred for another 1 h. Water and ethyl acetate were added and the mixture was filtrated on a celite pad. The layers were separated, and the aqueous phase was extracted twice with ethyl acetate. The combined organic layers were washed with an aqueous solution of brine, dried over Na₂SO₄, filtered, and concentrated to dryness. The crude product was purified by flash chromatography (Silica, HP-Sil 100 g column, 0-10% methanol in dichloromethane) and re-purified by flash chromatography (Silica, HP-Sil 100 g column, 40-100% ethyl acetate in heptane) to afford N-((4-bromo-1H-pyrrol-2-yl)methyl)pyridin-3-amine as an orange solid (1.19 g, 55%).

¹H NMR (80 MHz, DMSO-d6) δ 11.06 (s, 1H), 8.00 (d, 1H), 7.77 (dd, 1H), 7.18-6.91 (m, 2H), 6.77 (t, 1H), 6.23-5.94 (m, 2H), 4.14 (d, 2H).

MS: 253.94 [M+H]⁺

### Step B:

In a flask under argon, the compound from step A (250 mg, 0.992 mmol), (R)-2-(3-fluoropyrrolidin-1-yl)-5-(4,4,5,5-tetramethyl-1,3,2-dioxaborolan-2-yl)pyridine (319 mg, 1.091 mmol), bis(tri-tert-butylphosphine)palladium(0) (50.7 mg, 0.099 mmol) and cesium carbonate (969 mg, 2.97 mmol) were added, followed by a mixture of dry 1,4-dioxane (16 mL)/degassed water (4 mL). The reaction mixture was stirred at 85° C. for 4 h. The crude product was evaporated under reduced pressure, dissolved with water and extracted twice with a mixture of dichloromethane/methanol (9:1). The organic layers were combined, dried over Na₂SO₄, filtered and concentrated. The crude product was triturated in ethyl acetate, and the solid was triturated in dichloromethane to afford (R)—N-((4-(6-(3-fluoropyrrolidin-1-yl)pyridin-3-yl)-1H-pyrrol-2-yl)methyl)pyridin-3-amine as a dark brown solid (80.1 mg, 24%).

¹H NMR (80 MHz, DMSO-d6) δ 10.62 (s, 1H), 8.28 (d, 1H), 8.03 (s, 1H), 7.86-7.52 (m, 2H), 7.13-6.89 (m, 2H), 6.74-6.22 (m, 3H), 6.06 (t, 1H), 5.43 (d, 1H), 4.19 (s, 2H), 3.92-3.42 (m, 4H), 2.23-1.81 (m, 2H).

MS: 339.10 [M+H]⁺

### Step C:

The compound from step B (40 mg, 0.119 mmol) was dissolved in dichloroethane (3 mL) and 1,1′-carbonyldiimidazole (192 mg, 1.186 mmol) was added. The mixture was stirred at RT. 24 h later, 1,1′-carbonyldiimidazole (192 mg, 1.186 mmol was added. The mixture was stirred for 24 h to achieve completion. The crude reaction mixture was filtered and triturated in water to afford (R)-6-(6-(3-fluoropyrrolidin-1-yl)pyridin-3-yl)-2-(pyridin-3-yl)-1H-pyrrolo[1,2-c]imidazol-3(2H)-one as a brown solid (25.2 mg, 58.5%).

¹H NMR (80 MHz, DMSO-d6) δ 8.96 (d, 1H), 8.53-8.32 (m, 2H), 8.19 (dd, 1H), 7.85 (dd, 1H), 7.64 (s, 1H), 7.48 (dd, 1H), 6.71-6.39 (m, 2H), 5.91-4.89 (m, 3H), 3.75-3.35 (m, 4H), 2.23-1.80 (m, 2H).

MS: 364.12 [M+H]⁺

### Step D:

To a solution of the compound from step C (25 mg, 0.069 mmol) in dioxane (7 mL) at RT was added 4M hydrochloric acid in dioxane (0.5 mL, 2.00 mmol). The mixture was stirred at RT for 22 h. The solvent was evaporated, and the solid was triturated with acetonitrile, followed by ethyl acetate to afford (R)-6-(6-(3-fluoropyrrolidin-1-yl)pyridin-3-yl)-2-(pyridin-3-yl)-1H-pyrrolo[1,2-c]imidazol-3(2H)-one hydrochloride as a brown solid (17.1 mg, 62%).

¹H NMR (80 MHz, DMSO-d6) δ 9.07 (d, 1H), 8.60-8.19 (m, 4H), 7.99 (s, 1H), 7.70 (dd, 1H), 7.17 (d, 1H), 6.80 (s, 1H), 5.57 (d, 1H), 5.10 (s, 2H), 4.21-3.35 (m, 4H), 2.33-1.84 (m, 2H).

MS: 261.03 [M+H]⁺

### Example 2: (R)-2-(6-(3-fluoropyrrolidin-1-yl)pyridin-3-yl)-6-(pyridin-3-yl)-6,7-dihydro-5H-imidazo[1,5-a]imidazol-5-one Hydrochloride

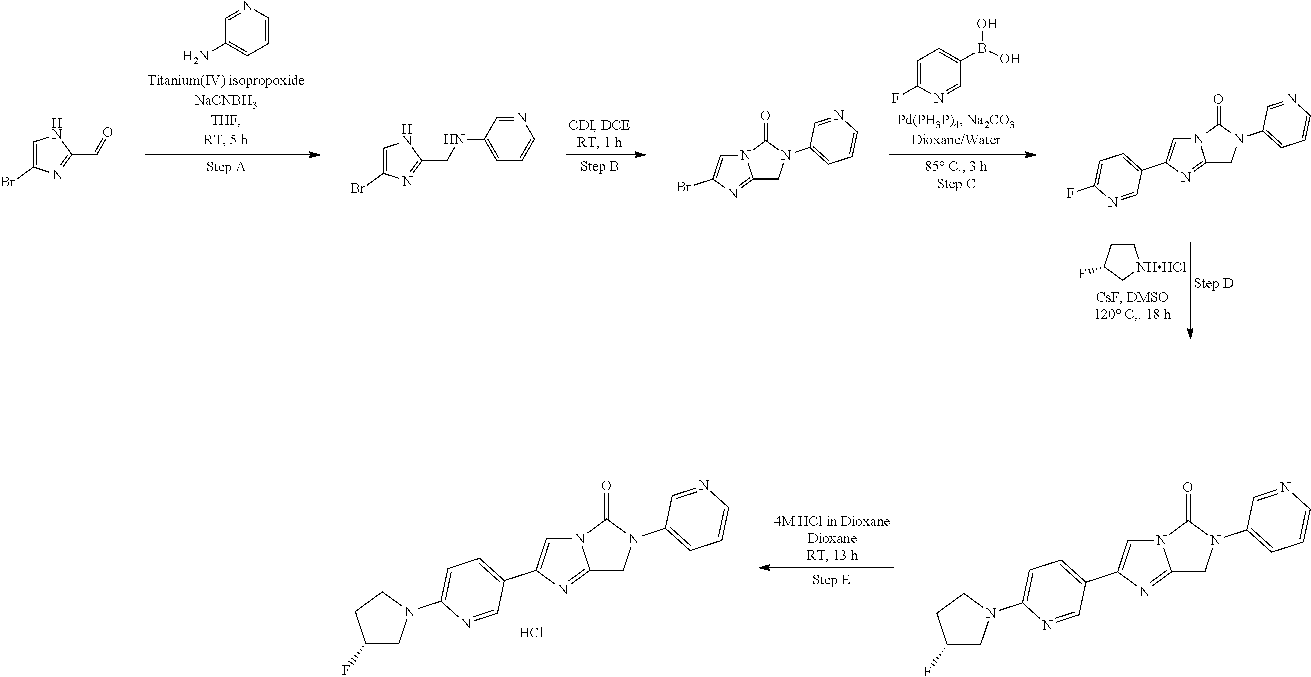

### Step A:

To a solution of 4-bromo-1H-imidazole-2-carbaldehyde (1 g, 5.71 mmol) in tetrahydrofuran (60 mL) was added titanium (IV) isopropoxide (6.70 mL, 22.86 mmol) followed by 3-aminopyridine (1.614 g, 17.14 mmol). The mixture was stirred at RT for 1 h30 before addition of titanium (IV) isopropoxide (6.70 mL, 22.86 mmol). After 1 h, sodium cyanoborohydride (0.813 g, 12.93 mmol) was added and the reaction was finished 2 h later. Water and ethyl acetate were added and the mixture was filtrated on a celite pad. The layers were separated, and the aqueous phase was extracted twice with ethyl acetate. The combined organic layers were washed with an aqueous solution of brine, dried over Na₂SO₄, filtered, and concentrated to dryness. The crude product was triturated in ethyl acetate to afford N-((4-bromo-1H-imidazol-2-yl)methyl)pyridin-3-amine as a pale pink solid (460 mg, 32%).

¹H NMR (80 MHz, DMSO-d6) δ 12.29 (s, 1H), 8.00 (d, 1H), 7.78 (dd, 1H), 7.16 (s, 1H), 7.11-6.90 (m, 2H), 6.35 (t, 1H), 4.24 (d, 2H).

MS: 254.97 [M+H]⁺

### Step B:

The compound from step A (460 mg, 1.817 mmol) was dissolved in dichloroethane (50 mL) and 1,1′-carbonyldiimidazole (2947 mg, 18.17 mmol) was added. The mixture was stirred at RT for 1 h. The solvent was evaporated and the crude product was purified by flash chromatography (Silica, HP-Sil 100 g column, 0-10% methanol in dichloromethane) to afford 2-bromo-6-(pyridin-3-yl)-6,7-dihydro-5H-imidazo[1,5-a]imidazol-5-one as a white solid (412 mg, 81%).

¹H NMR (80 MHz, DMSO-d6) δ 8.94 (d, 1H), 8.44 (dd, 1H), 8.25-8.05 (m, 1H), 7.97 (s, 1H), 7.50 (dd, 1H), 5.10 (s, 2H).

MS: 280.95 [M+H]⁺

### Step C:

In a flask under argon, the compound from step B (275 mg, 0.985 mmol), (6-fluoropyridin-3-yl)boronic acid (208 mg, 1.478 mmol), tetrakis(triphenylphosphine)palladium(0) (171 mg, 0.148 mmol) and sodium carbonate (209 mg, 1.971 mmol) were added to a mixture of dry 1,4-dioxane (10 mL)/degassed water (2.50 mL). The mixture was heated at 85° C. for 3 h. The crude product was evaporated under reduced pressure, dissolved with water and extracted three times with dichloromethane. The organic layers were combined, dried over Na₂SO₄, filtered and concentrated. The crude product was triturated in ethyl acetate to afford 2-(6-fluoropyridin-3-yl)-6-(pyridin-3-yl)-6,7-dihydro-5H-imidazo[1,5-a]imidazol-5-one as a white solid (128.8 mg, 44.3%).

¹H NMR (80 MHz, DMSO-d6) δ 8.98 (d, J=2.7 Hz, 1H), 8.75 (d, J=1.5 Hz, 1H), 8.57-8.33 (m, 3H), 8.20 (d, J=8.9 Hz, 1H), 7.52 (dd, J=8.4, 4.7 Hz, 1H), 7.28 (dd, J=8.6, 2.8 Hz, 1H), 5.17 (s, 2H).

MS: 296.08 [M+H]⁺

### Step D:

In a vial under argon, compound from step C (40 mg, 0.135 mmol), (R)-3-fluoropyrrolidine hydrochloride (68.0 mg, 0.542 mmol), and cesium fluoride (123 mg, 0.813 mmol) were mixed in dry dimethylsulfoxide (3 mL). The mixture was flushed with argon and stirred at 120° C. for 18 h. The reaction mixture was cooled down and poured into cold water pre-cooled in an ice bath. The resulting solution was filtered, the solid was rinsed with water and isopropanol. The solid was dried to afford (R)-2-(6-(3-fluoropyrrolidin-1-yl)pyridin-3-yl)-6-(pyridin-3-yl)-6,7-dihydro-5H-imidazo[1,5-a]imidazol-5-one as a white solid (36.6 mg, 74%)

¹H NMR (400 MHz, DMSO-d6) δ 8.97 (d, 1H), 8.64 (s, 1H), 8.42 (d, J=4.5 Hz, 1H), 8.18 (d, J=8.5 Hz, 1H), 8.06 (s, 1H), 7.99 (d, J=8.7 Hz, 1H), 7.52 (dd, J=7.3 Hz, 1H), 6.57 (d, J=9.0 Hz, 1H), 5.46 (d, J=53.6 Hz, 1H), 5.12 (s, 2H), 3.87-3.51 (m, 4H), 2.18 (d, J=77.1 Hz, 2H).

MS: 365.22 [M+H]⁺

### Step E:

To a solution of the compound from step D (35 mg, 0.096 mmol) in dioxane (7 mL) at RT was added 4M hydrochloric acid in dioxane (0.7 ml, 2.80 mmol). The mixture was stirred at RT for 13 h. The solvent was evaporated to afford (R)-2-(6-(3-fluoropyrrolidin-1-yl)pyridin-3-yl)-6-(pyridin-3-yl)-6,7-dihydro-5H-imidazo[1,5-a]imidazol-5-one hydrochloride as a beige solid (45.4 mg, 118%).

¹H NMR (80 MHz, DMSO-d6) δ 9.09 (d, J=2.6 Hz, 1H), 8.64-8.29 (m, 5H), 7.74 (dd, J=8.7, 4.8 Hz, 1H), 7.24 (d, J=9.2 Hz, 1H), 6.01-5.08 (m, 3H), 3.99-3.52 (m, 4H), 2.22-1.81 (m, 2H).

MS: 365.19 [M+H]⁺

### Example 3: (S)-2-(6-(3-fluoropyrrolidin-1-yl)pyridin-3-yl)-6-(pyridin-3-yl)-6,7-dihydro-5H-imidazo[1,5-a]imidazol-5-one Hydrochloride

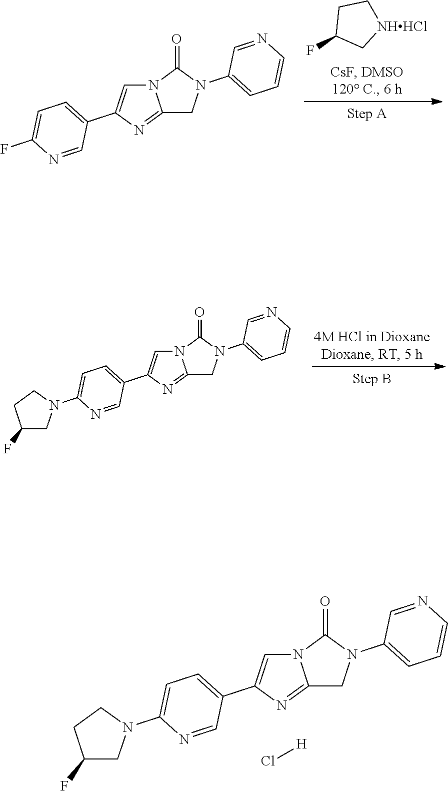

### Step A:

In a vial under argon, the compound from Example 2 step C (40 mg, 0.135 mmol), (S)-3-fluoropyrrolidine hydrochloride (68.0 mg, 0.542 mmol), and cesium fluoride (123 mg, 0.813 mmol) were mixed in dry dimethylsulfoxide (3 mL). The mixture was flushed with argon and stirred at 120° C. for 6 h. The reaction mixture was cooled down and poured into cold water pre-cooled in an ice bath. The resulting solution was filtered, the solid was rinsed with water and isopropanol. The solid was dried to afford (S)-2-(6-(3-fluoropyrrolidin-1-yl)pyridin-3-yl)-6-(pyridin-3-yl)-6,7-dihydro-5H-imidazo[1,5-a]imidazol-5-one as a beige solid (32.9 mg, 67%)

¹H NMR (80 MHz, DMSO-d6) δ 8.95 (d, 1H), 8.62 (d, 1H), 8.41 (dd, J=4.4 Hz, 1H), 8.19 (d, 1H), 8.08-7.88 (m, 2H), 7.50 (dd, J=8.3, 5.0 Hz, 1H), 6.57 (d, J=9.0 Hz, 1H), 5.89-5.02 (m, 3H), 3.97-3.44 (m, 4H), 2.32-1.91 (m, 2H).

MS: 365.15 [M+H]⁺

### Step B:

To a solution of the compound from step D (32.9 mg, 0.090 mmol) in dioxane (7 mL) at RT was added 4M hydrochloric acid in dioxane (0.7 mL, 2.80 mmol). The mixture was stirred at RT for 5 h. The solvent was evaporated to afford (S)-2-(6-(3-fluoropyrrolidin-1-yl)pyridin-3-yl)-6-(pyridin-3-yl)-6,7-dihydro-5H-imidazo[1,5-a]imidazol-5-one hydrochloride as a beige solid (41.3 mg, 114%).

¹H NMR (80 MHz, DMSO-d6) δ 9.04 (d, J=2.7 Hz, 1H), 8.60-8.21 (m, 5H), 7.64 (dd, J=8.4, 4.8 Hz, 1H), 7.19 (d, J=9.5 Hz, 1H), 6.00-5.07 (m, 3H), 4.47-3.93 (m, 4H), 2.29-1.84 (m, 2H).

MS: 365.20 [M+H]⁺

### Example 4: 6-(6-fluoropyridin-3-yl)-2-(pyridin-3-yl)-1H-pyrrolo[1,2-c]imidazol-3(2H)-one

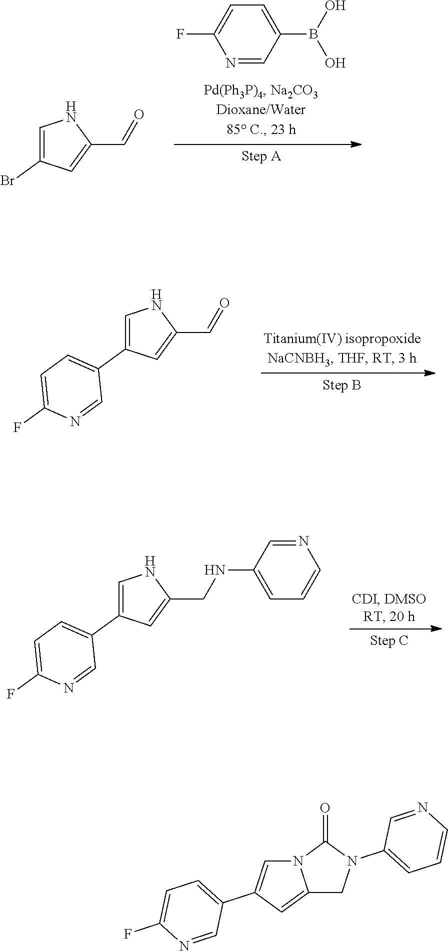 [SMILES: OB(O)c1ccc(F)nc1 ; O=Cc1cc(Br)c[nH]1 ; O=Cc1cc(-c2ccc(F)nc2)c[nH]1 ; Fc1ccc(-c2c[nH]c(CNc3cccnc3)c2)cn1 ; O=C1N(c2cccnc2)Cc2cc(-c3ccc(F)nc3)cn21]

### Step A:

In a flask under argon, 4-bromo-1H-pyrrole-2-carbaldehyde (5 g, 28.7 mmol), (6-fluoropyridin-3-yl)boronic acid (6.07 g, 43.1 mmol), tetrakis(triphenylphosphine)palladium(0) (4.98 g, 4.31 mmol) and sodium carbonate (6.09 g, 57.5 mmol) were added to a mixture of dry 1,4-dioxane (100 mL)/degassed water (25 mL). The mixture was heated at 85° C. for 3 h before addition of tetrakis(triphenylphosphine)palladium(0) (4.98 g, 4.31 mmol). The mixture was stirred at 85° C. overnight. The crude product was evaporated under reduced pressure, dissolved with water and extracted three times with dichloromethane. The organic layers were combined, dried over Na₂SO₄, filtered and concentrated. The crude product was triturated in dichloromethane, the filtrate evaporated and triturated in ethyl acetate. The filtrate was triturated once more in dichloromethane and the solids combined to afford 4-(6-fluoropyridin-3-yl)-1H-pyrrole-2-carbaldehyde as a white solid (730 mg, 13%)

¹H NMR (80 MHz, DMSO-d6) δ 12.40 (s, 1H), 9.54 (s, 1H), 8.56 (d, 1H), 8.24 (td, 1H), 7.84 (s, 1H), 7.48 (d, 1H), 7.18 (dd, 1H).

MS: 191.00 [M+H]⁺

### Step B:

To a solution of compound from step A (500 mg, 2.63 mmol), in tetrahydrofuran (25 mL) was added titanium (IV) isopropoxide (1.541 mL, 5.26 mmol), followed by 3-aminopyridine (247 mg, 2.63 mmol). The mixture was stirred at RT for 1 h before addition of 1 mL of titanium (IV) isopropoxide and 15 min later a spatula tip of 3-aminopyridine was added. Sodium cyanoborohydride (330 mg, 5.26 mmol) was added 40 min later. Water and ethyl acetate were added and the mixture was filtrated on a celite pad. The layers were separated, and the aqueous phase was extracted twice with ethyl acetate. The combined organic layers were washed with an aqueous solution of brine, dried over Na₂SO₄, filtered, and concentrated to dryness. The solid crude product was successively triturated in ethyl acetate, dichloromethane and acetonitrile to afford N-((4-(6-fluoropyridin-3-yl)-1H-pyrrol-2-yl)methyl)pyridin-3-amine as a pale yellow solid (359 mg, 50.9%).

¹H NMR (80 MHz, DMSO-d6) δ 11.06 (s, 1H), 8.36 (d, 1H), 8.20-7.91 (m, 2H), 7.77 (dd, 1H), 7.31-6.93 (m, 4H), 6.42 (d, 1H), 6.14 (t, 1H), 4.19 (d, 2H).

MS: 269.03 [M+H]⁺

### Step C:

The compound from step B (140 mg, 0.522 mmol) was dissolved in dimethylsulfoxide (20 mL) and 1,1′-carbonyldiimidazole (846 mg, 5.22 mmol). The mixture was stirred at RT for 20 h. The reaction mixture was cooled down and poured into cold water pre-cooled in an ice bath. The resulting solution was filtered, and the solid was rinsed with water. The solid was dried to afford 6-(6-fluoropyridin-3-yl)-2-(pyridin-3-yl)-1H-pyrrolo[1,2-c]imidazol-3(2H)-one as a beige solid (97.6 mg, 63.6%).

¹H NMR (80 MHz, DMSO-d6) δ 8.96 (d, J=2.7 Hz, 1H), 8.61 (d, 1H), 8.47-8.08 (m, 3H), 7.93 (s, 1H), 7.49 (dd, J=8.5, 4.7 Hz, 1H), 7.21 (dd, J=8.5, 3.0 Hz, 1H), 6.75 (d, J=1.5 Hz, 1H), 5.08 (s, 2H).

MS: 295.02 [M+H]⁺

### Example 5: (S)-6-(6-(3-fluoropyrrolidin-1-yl) pyridin-3-yl)-2-(1-methyl-1H-pyrazol-4-yl)-1,2-dihydro-3H-pyrrolo[1,2-c]imidazol-3-one Hydrogen Chloride Salt

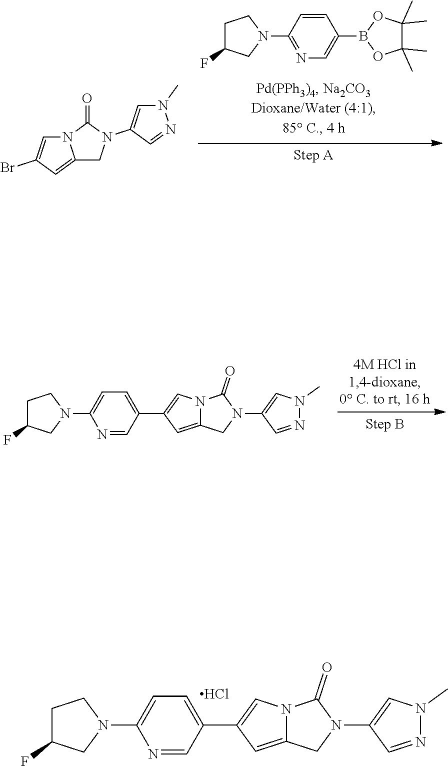

### Step A:

In an oven-dried screw capped vial was added 6-bromo-2-(1-methyl-1H-pyrazol-4-yl)-1,2-dihydro-3H-pyrrolo[1,2-c]imidazol-3-one (150 mg, 0.53 mmol), boronic ester (311 mg, 1.06 mmol), Na₂CO₃ (170 mg, 1.6 mmol) and (1,4-dioxane:H₂O) (4:1, 7.5 mL) under an argon atmosphere. The reaction mixture was degassed with argon for 15 min. Then, Pd(PPh₃)₄ (62 mg, 0.053 mmol) was added and the mixture was heated to 85° C. for 5 h. The reactants were consumed as monitored by TLC. After that the reaction mixture was quenched with ice-water (10 mL) and extracted with 5% MeOH in DCM (30 mL×3). The organic layer was dried over Na₂SO₄, concentrated and purified by silica gel chromatography (100-200 mesh) eluted in 2% MeOH in DCM to get (S)-6-(6-(3-fluoropyrrolidin-1-yl) pyridin-3-yl)-2-(1-methyl-1H-pyrazol-4-yl)-1,2-dihydro-3H-pyrrolo[1,2-c]imidazol-3-one as an off-white solid (40 mg, 20%).

¹H NMR (DMSO-d6) δ 8.44 (d, 1H), 7.98 (s, 1H), 7.83 (dd, 1H), 7.64 (d, 1H), 7.60 (d, 1H), 6.56 (m, 1H), 6.52 (d, 1H), 5.45 (m, 1H), 4.81 (s, 2H), 3.86 (s, 3H), 3.66 (m, 3H), 3.44 (m, 1H), 2.20 (m, 2H).

LCMS: 366.9 [M]+

### Step B:

To a stirred solution of the compound from step A (40 mg, 0.11 mmol) in 1,4-dioxane (1.2 mL) was added 4M HCl in 1,4-dioxane (0.2 mL) at 0° C. under N₂ atmosphere and the mixture was stirred at RT for 16 h. After completion of the reaction, solvent was evaporated, the residue was washed with pentane, and dried under vacuum to afford (S)-6-(6-(3-fluoropyrrolidin-1-yl) pyridin-3-yl)-2-(1-methyl-1H-pyrazol-4-yl)-1,2-dihydro-3H-pyrrolo[1,2-c]imidazol-3-one hydrogen chloride salt as a white solid (10 mg, 25%).

¹H NMR (DMSO-d6) δ 8.28 (d, 2H), 8.02 (s, 1H), 7.89 (s, 1H), 7.66 (s, 1H), 7.08 (s, 1H), 6.72 (s, 1H), 5.55 (d, 1H), 4.84 (s, 2H), 3.84 (m, 7H), 2.25 (m, 2H).

LCMS: 366.9 [M]+

### Example 6: (R)-6-(6-(3-fluoropyrrolidin-1-yl) pyridin-3-yl)-2-(1-methyl-1H-pyrazol-4-yl)-1,2-dihydro-3H-pyrrolo[1,2-c]imidazol-3-one Hydrogen Chloride Salt

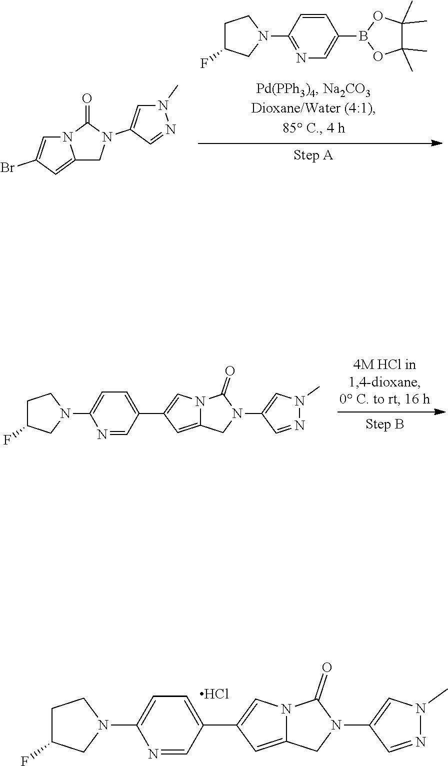

### Step A:

In an oven-dried screw capped vial was added 6-bromo-2-(1-methyl-1H-pyrazol-4-yl)-1,2-dihydro-3H-pyrrolo[1,2-c]imidazol-3-one (150 mg, 0.53 mmol), boronic ester (311 mg, 1.1 mmol), Na₂CO₃ (170 mg, 1.6 mmol) and dioxane:H₂O (4:1, 7.5 mL) under an argon atmosphere. The reaction mixture was degassed with argon for 15 min. Then, Pd(PPh₃)₄ (62 mg, 0.053 mmol) was added and the mixture was heated to 85° C. for 4 h. The reactants were consumed as monitored by TLC. After that the reaction mixture was quenched with ice-water (15 mL) and extracted with 5% MeOH in DCM (15 mL×3). The organic layer was dried over Na₂SO₄, concentrated and purified by silica gel chromatography (100-200 mesh) eluted in 3% MeOH in DCM to get (R)-6-(6-(3-fluoropyrrolidin-1-yl) pyridin-3-yl)-2-(1-methyl-1H-pyrazol-4-yl)-1,2-dihydro-3H-pyrrolo[1,2-c]imidazol-3-one as a yellow solid (40 mg, 20%).

¹H NMR (DMSO-D6) δ 8.44 (d, 1H), 7.98 (s, 1H), 7.83 (dd, 1H), 7.61 (d, 2H), 6.54 (m, 2H), 5.45 (m, 1H), 4.81 (s, 2H), 3.86 (s, 3H), 3.66 (m, 3H), 3.44 (m, 1H), 2.20 (m, 2H).

LCMS: 366.9 (M)+

### Step B:

To a stirred solution of the compound from step A (40 mg, 0.1 mmol) in DCM (0.8 mL) was added 4M HCl in 1,4-dioxane (0.2 mL) at 0° C. under N₂ atmosphere and the mixture was stirred at rt for 16 h. Then, solvent was evaporated, the residue was washed with pentane, and dried under vacuum to afford (R)-6-(6-(3-fluoropyrrolidin-1-yl) pyridin-3-yl)-2-(1-methyl-1H-pyrazol-4-yl)-1,2-dihydro-3H-pyrrolo[1,2-c]imidazol-3-one hydrogen chloride salt as a white solid (40 mg, 90%).

¹H NMR (DMSO-d6) δ 8.40 (dd, 1H), 8.20 (d, 1H), 8.00 (s, 1H), 7.94 (s, 1H), 7.65 (s, 1H), 7.20 (d, 1H), 6.74 (d, 1H), 5.57 (d, 1H), 4.85 (s, 2H), 3.94 (m, 2H), 3.86 (s, 3H), 3.67 (m, 2H), 2.30 (m, 2H).

LCMS: 366.9 (M)+

### Example 7: (S)-6-(6-(3-fluoropyrrolidin-1-yl) pyridin-3-yl)-2-(pyridin-3-yl)-1,2-dihydro-3H-pyrrolo[1,2-c]imidazol-3-one Hydrogen Chloride Salt

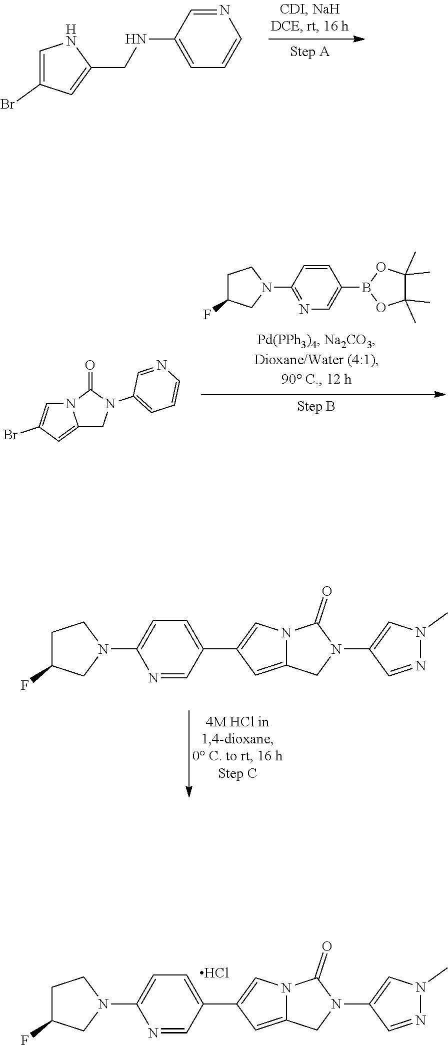 [SMILES: Brc1c[nH]c(CNc2cccnc2)c1 ; CC1(C)OB(c2ccc(N3CC[C@H](F)C3)nc2)OC1(C)C ; O=C1N(c2cccnc2)Cc2cc(Br)cn21 ; Cn1cc(N2Cc3cc(-c4ccc(N5CC[C@H](F)C5)nc4)cn3C2=O)cn1 ; Cn1cc(N2Cc3cc(-c4ccc(N5CC[C@H](F)C5)nc4)cn3C2=O)cn1 ; Cl]

### Step A:

To an ice cool solution of example 1 step A (500 mg, 1.9 mmol) in 1,2-DCE (5.0 mL) was added NaH (60% dispersed in mineral oil) (24 mg, 0.98 mmol) under N₂ atmosphere. Then, the mixture was allowed to warm to RT and was kept for 30 min. Then, CDI (3.2 g, 19.6 mmol) was added to the reaction mixture and the mixture was stirred at RT for 16 h. The reaction mixture was quenched with ice cold water and the product was extracted with EtOAc (40 mL×3). The extract was dried over Na₂SO₄ and concentrated under vacuum. The residue was purified by silica gel chromatography (100-200 mesh) eluted in 20% EtOAc in hexane to yield 6-bromo-2-(pyridin-3-yl)-1,2-dihydro-3H-pyrrolo[1,2-c]imidazol-3-one as a yellow solid (250 mg, 45%).

¹H NMR (DMSO-d6) δ 8.92 (d, 1H), 8.40 (d, 1H), 8.15 (m, 1H), 7.50 (m, 2H), 6.34 (s, 1H), 5.03 (s, 2H).

MS (ESI): 280.15 (M+H)+;

### Step B:

In an oven-dried screw capped vial was added the compound from step A (100 mg, 0.35 mmol), boronic ester (210 mg, 0.72 mmol), Cs₂CO₃ (233 mg, 0.72 mmol) and dioxane:H₂O (4:1, 5 mL) under an argon atmosphere. The reaction mixture was degassed with argon for 15 min. Then, Pd(dppf)Cl₂·DCM (30 mg, 0.036 mmol) was added and the mixture was heated to 90° C. for 12 h. The reactants were consumed as monitored by TLC. After that the reaction mixture was quenched with ice-water (15 mL) and extracted with 5% MeOH in DCM (50 mL×3). The organic layer was dried over Na₂SO₄, concentrated and purified by silica gel chromatography (100-200 mesh) eluted in 4% MeOH in DCM to get (S)-6-(6-(3-fluoropyrrolidin-1-yl) pyridin-3-yl)-2-(pyridin-3-yl)-1,2-dihydro-3H-pyrrolo[1,2-c]imidazol-3-one as an off-white solid (25 mg, 19%).

¹H NMR (DMSO-d6) δ 8.96 (d, 1H), 8.47 (d, 1H), 8.39 (d, 1H), 8.19 (d, 1H), 7.86 (dd, 1H), 7.65 (s, 1H), 7.49 (q, 1H), 6.61 (s, 1H), 6.53 (d, 1H), 5.46 (d, 1H), 5.05 (s, 2H), 3.67 (m, 3H), 3.44 (m, 1H), 2.21 (m, 2H).

LCMS: 364.0 (M+H)+

### Step C:

To a stirred solution of the compound from step B (25 mg, 0.07 mmol) in DCM (0.7 mL) was added 4M HCl in 1,4-dioxane (0.12 mL) at 0° C. under N₂ atmosphere and the mixture was stirred at RT for 10 h. After completion of the reaction, solvent was evaporated, the residue was washed with pentane, and dried under vacuum to afford (S)-6-(6-(3-fluoropyrrolidin-1-yl) pyridin-3-yl)-2-(pyridin-3-yl)-1,2-dihydro-3H-pyrrolo[1,2-c]imidazol-3-one hydrogen chloride salt as a white solid (15 mg, 55%).

¹H NMR (DMSO-d6) δ 8.50 (d, 1H), 8.42 (d, 1H), 8.24 (s, 1H), 8.03 (s, 1H), 7.97 (s, 1H), 7.20 (d, 1H), 6.82 (s, 1H), 5.58 (d, 1H), 5.11 (s, 2H), 3.90 (m, 3H), 3.67 (td, 1H), 2.30 (m, 2H).

LCMS: 363.9 (M)+

### Biological Assay Description and Corresponding Results

1. Preparation of Human Parkinson's Disease (PD) Brain-Derived Alpha-Synuclein (a-Syn) Aggregates

The procedure was adapted from the protocol described in Spillantini et al., 1998. Frozen tissue blocks from PD donors were thawed on ice and homogenized using a glass dounce homogenizer. The homogenate was then centrifuged at 11,000×g (12,700 RPM) in an ultracentrifuge (Beckman, XL100K) for 20 minutes at 4° C. using a pre-cooled 70.1 rotor (Beckman, 342184). Pellets were resuspended in extraction buffer [10 mM Tris-HCl pH 7.4, 10% sucrose, 0.85 M NaCl, 1% protease inhibitor (Calbiochem 539131), 1 mM EGTA, 1% phosphatase inhibitor (Sigma P5726 and P0044)] and centrifuged at 15,000×g (14,800 RPM, a 70.1 Ti rotor) for 20 minutes at 4° C. Pellets were discarded and sarkosyl (20% stock solution, Sigma L7414) was added to the supernatants to a final concentration of 1% and stirred at room temperature for one hour. This solution was then centrifuged at 100,000×g (38,000 RPM, 70.1 Ti rotor) for one hour at 4° C. Pellets containing enriched alpha-synuclein aggregates were resuspended in PBS per gram of brain initially used and stored at −80° C. until use.

### 2. Micro-Radiobinding Competition Assay for the Determination of Binding Affinity

PD brain-derived alpha-synuclein aggregates were spotted onto microarray slides. The slides were incubated with [³H]-alpha-synuclein reference at 20 nM or 30 nM and the example compounds (non-radiolabelled) at 1 μM and 100 nM. In some cases, the non-radiolabelled example compounds were further assessed for a range of different concentrations, varying from 0.05 nM to 2 μM. After incubation, slides were washed and scanned by a real-time autoradiography system (BeaQuant, ai4R). Quantification of signal was performed by using the image analysis software Beamage (ai4R).

Non-specific signal was determined with an excess of non-radiolabelled alpha-syn reference compound (2 μM) and specific binding was calculated by subtracting the non-specific signal from the total signal. Competition was calculated as percentage, where 0% was defined as the specific binding in the presence of vehicle and 100% as the values obtained in the presence of excess of the non-radiolabelled alpha-syn reference compound. Kᵢ values were calculated in GraphPad Prism7 by applying a nonlinear regression curve fit using a one site, specific binding model. All measurements were performed with at least two technical replicates. For compounds tested in more than one experiment, the mean of the replicates or Kᵢ values in independent experiments is reported.

### Results

Example compounds were assessed for their potency to compete with the binding of [³H]-reference alpha-synuclein ligand to PD patient brain-derived alpha-synuclein aggregates. Results of the micro-radiobinding competition assay for the example compounds tested are shown in Table 3 as: % competition at 1 μM and 100 nM. Table 3 also shows Kᵢ values.

TABLE 3

| Example | Micro-radiobinding competition assay | | |
|---|---|---|---|
| Compound | Competition at | Competition | |
| no. | 1 μM (%) | at 100 nM (%) | Ki (nM) |
| 1 | 95 | 75 | n.d. |
| 2 | 86 | 63 | 124 |
| 3 | 85 | 59 | 97 |
| 4 | 52 | 51 | n.d. |
| 5 | 102 | 87 | n.d. |
| 6 | 100 | 82 | n.d. |
| 7 | 85 | 69 | n.d. |

Table 3: Assessment of binding affinity by micro-radiobinding competition assay on human PD brain-derived alpha-synuclein aggregates. Percent (%) competition over the tritiated [³H]-alpha-syn reference ligand in the presence of 1 μM and 100 nM of example compounds 1-7. Kᵢ values are also shown for selected example compounds. The examples are the mean of Ki values in two independent experiments using PD brain-derived homogenates from two different donors. As shown in Table 3, example compounds 1-7 of the present invention show potent binding to PD brain-derived alpha-synuclein aggregates. (n.d.=not determined)

1. A compound of formula (I): 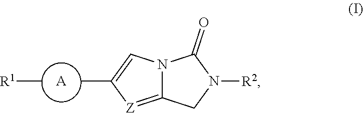
or a detectably labelled compound, stereoisomer, racemic mixture, pharmaceutically acceptable salt, hydrate, or solvate thereof, wherein 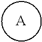
is a 6-membered heteroaryl, which is optionally substituted with at least one halo, haloC₁-C₄alkyl, haloC₁-C₄alkoxy, C₁-C₄alkoxy, or C₁-C₄alkyl;
R¹ is a 4- to 6-membered heterocyclyl which is optionally substituted with at least one halo; or
R¹ is halo, haloC₁-C₄alkyl, haloC₁-C₄alkoxy, C₁-C₄alkoxy, or C₁-C₄alkyl; or
R¹ is —NH—C₃-C₆cycloalkyl or —C₃-C₆cycloalkyl, wherein the C₃-C₆cycloalkyl of —NH—C₃-C₆cycloalkyl or —C₃-C₆cycloalkyl is optionally substituted with at least one halo;
R² is a 5-membered or 6-membered heteroaryl, optionally substituted with 1 or 2 substituents independently selected from haloC₁-C₄alkyl, haloC₁-C₄alkoxy, C₁-C₄alkoxy, and C₁-C₄alkyl; and
Z is CH or N.

2. The compound according to claim 1, having a formula (Ia): 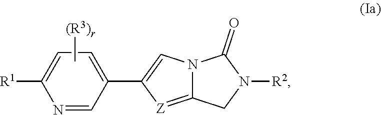
or a detectably labelled compound, stereoisomer, racemic mixture, pharmaceutically acceptable salt, hydrate, or solvate thereof,
wherein R³ is halo, haloC₁-C₄alkyl, haloC₁-C₄alkoxy, C₁-C₄alkoxy, or C₁-C₄alkyl; and
r is 0, 1 or 2.

3. The compound according to claim 1, wherein R¹ is a 4- to 6-membered heterocyclyl selected from the following: 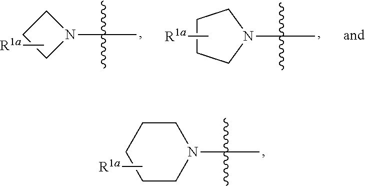
wherein R^{1a} is F;
or R¹ is halo; preferably wherein R¹ is a 5-membered heterocyclyl which is: 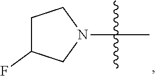 [SMILES: *N1CCC(F)C1]
or R¹ is F, preferably F is ¹⁹F or ¹⁸F, more preferably ¹⁸F.

4. (canceled)

5. The compound according to claim 1, wherein R² a 5-membered or 6-membered heteroaryl selected from the following: 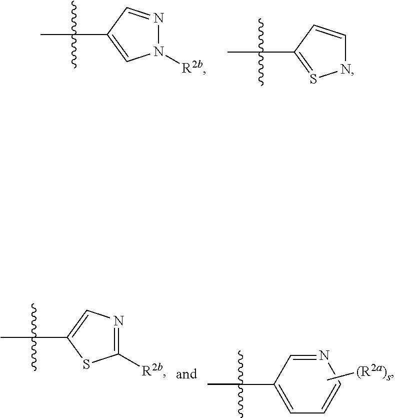
wherein
R^{2a} is selected from haloC₁-C₄alkyl, haloC₁-C₄alkoxy, C₁-C₄alkoxy, and C₁-C₄alkyl;
R^{2b} is selected from H, haloC₁-C₄alkyl, haloC₁-C₄alkoxy, C₁-C₄alkoxy, and C₁-C₄alkyl; and
s is 0, 1 or 2, preferably wherein R² a 5-membered or 6-membered heteroaryl selected from the following: 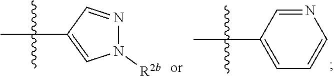
wherein
R^{2b} is selected from H, or C₁-C₄alkyl.

6. (canceled)

7. The compound according to claim 1, wherein the compound is selected from: 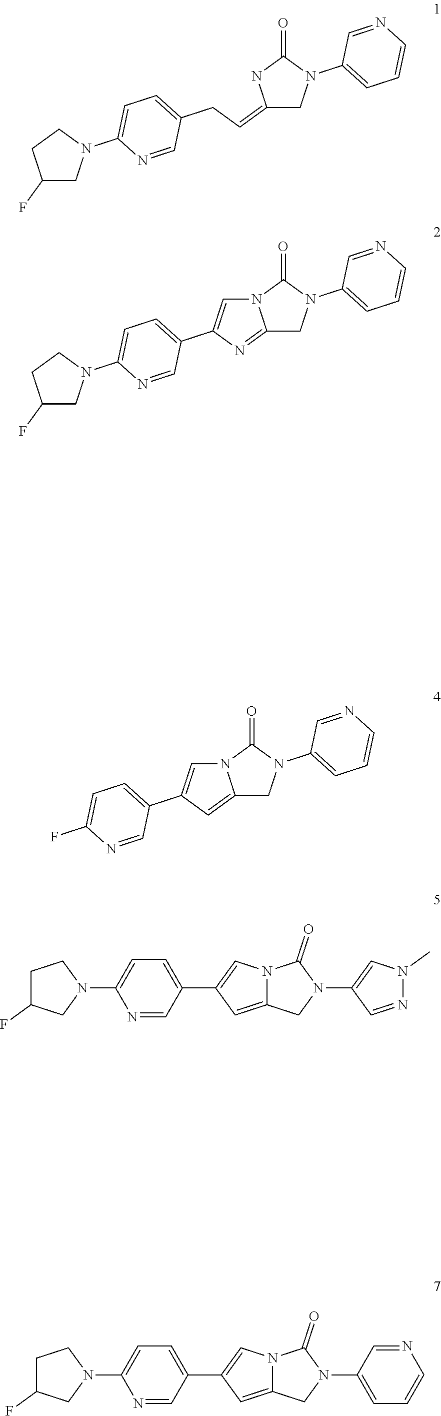 [SMILES: O=C1N/C(=C\Cc2ccc(N3CCC(F)C3)nc2)CN1c1cccnc1 ; O=C1N(c2cccnc2)Cc2nc(-c3ccc(N4CCC(F)C4)nc3)cn21 ; O=C1N(c2cccnc2)Cc2cc(-c3ccc(F)nc3)cn21 ; Cn1cc(N2Cc3cc(-c4ccc(N5CCC(F)C5)nc4)cn3C2=O)cn1 ; O=C1N(c2cccnc2)Cc2cc(-c3ccc(N4CCC(F)C4)nc3)cn21]
or a detectably labelled compound, stereoisomer, pharmaceutically acceptable salt, hydrate, or solvate thereof.

8. The compound according to claim 7, wherein the compound is selected from: 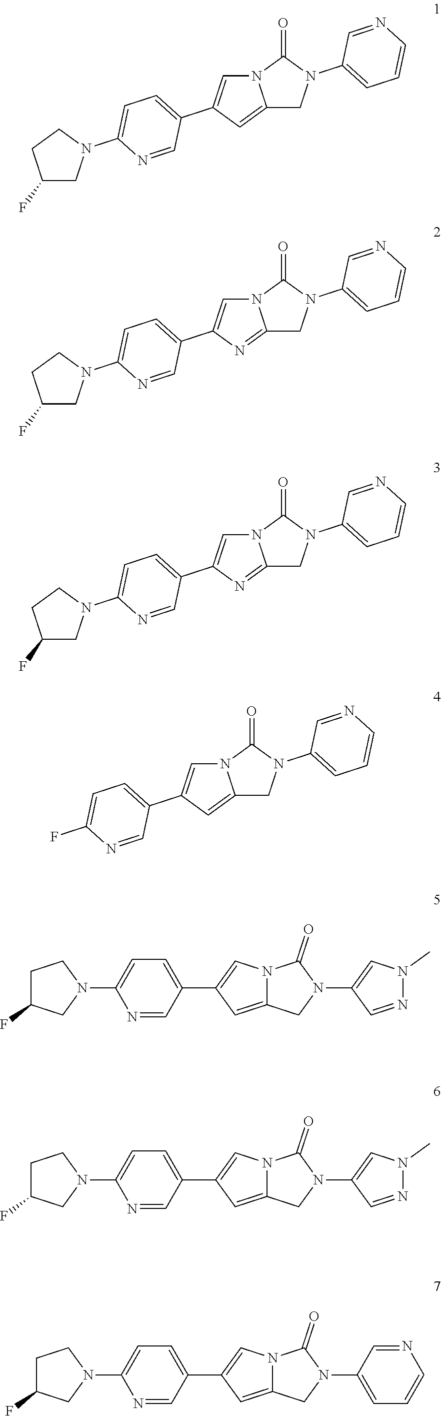
or a detectably labelled compound, pharmaceutically acceptable salt, hydrate, or solvate thereof.

9. The compound according to claim 1, wherein the compound is a detectably labelled compound preferably wherein the detectably labelled compound comprises a label selected from ¹⁸F, ²H and ³H.

10. (canceled)

11. The compound according to claim 9 wherein R¹ is 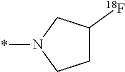 [SMILES: *N1CCC([18F])C1]
or ¹⁸F.

12. A diagnostic composition comprising a compound according to claim 9, and optionally at least one pharmaceutically acceptable excipient, carrier, diluent and/or adjuvant.

13. A method of imaging of alpha-synuclein aggregates, wherein the compound according to claim 9 is employed, particularly wherein the imaging is positron emission tomography imaging of alpha-synuclein aggregates, more particularly wherein the imaging is in vitro imaging, ex vivo imaging, or in vivo imaging, further particularly wherein the imaging is in vivo imaging, even more particularly the imaging is brain imaging.

14. (canceled)

15. (canceled)

16. (canceled)

17. A method of diagnosing a disease, disorder or abnormality associated with alpha-synuclein aggregates or a predisposition therefor, wherein the compound according to claim 1 or a detectably labelled compound, stereoisomer, racemic mixture, pharmaceutically acceptable salt, hydrate, or solvate thereof is employed, and wherein the disease, disorder or abnormality is optionally selected from Parkinson's disease (including sporadic, familial with alpha-synuclein mutations, familial with mutations other than alpha-synuclein, pure autonomic failure or Lewy body dysphagia), SNCA duplication carrier, Lewy Body dementia (LBD), dementia with Lewy bodies (DLB) (including “pure” Lewy body dementia), Parkinson's disease dementia (PDD), diffuse Lewy body disease (DLBD), Alzheimer's disease, sporadic Alzheimer's disease, familial Alzheimer's disease with APP mutations, familial Alzheimer's disease with PS-1, PS-2 or other mutations, familial British dementia, Lewy body variant of Alzheimer's disease, Down syndrome, multiple system atrophy (MSA) (including Shy-Drager syndrome, striatonigral degeneration or olivopontocerebellar atrophy), traumatic brain injury, chronic traumatic encephalopathy, dementia pugilistica, tauopathies (including Pick's disease, frontotemporal dementia, progressive supranuclear palsy, corticobasal degeneration, Niemann-Pick type C1 disease, frontotemporal dementia with Parkinsonism linked to chromosome 17), Creutzfeldt-Jakob disease, Huntington's disease, motor neuron disease, amyotrophic lateral sclerosis (including sporadic, familial or ALS-dementia complex of Guam), neuroaxonal dystrophy, neurodegeneration with brain iron accumulation type 1 (including Hallervorden-Spatz syndrome), prion diseases, ataxia telangiectatica, Meige's syndrome, subacute sclerosing panencephalitis, Gerstmann-Straussler-Scheinker disease, inclusion-body myositis, Gaucher disease, Krabbe disease as well as other lysosomal storage disorders (including Kufor-Rakeb syndrome and Sanfilippo syndrome) and rapid eye movement (REM) sleep behavior disorder.

18. The method according to claim 17, wherein the disease is Parkinson's disease, multiple system atrophy, dementia with Lewy bodies, Parkinson's disease dementia, SNCA duplication carrier, or Alzheimer's disease.

19. (canceled)

20. (canceled)

21. (canceled)

22. (canceled)

23. (canceled)

24. (canceled)

25. A method of diagnosing a disease, disorder or abnormality associated with alpha-synuclein aggregates, in a subject, the method comprising the steps:
(a) Administering a compound according to claim 1 to the subject;
(b) Allowing the compound to bind to the alpha-synuclein aggregates;
(c) Detecting the compound bound to the alpha-synuclein aggregates and
(d) Optionally generating an image representative of the location and/or amount of the compound bound to the alpha-synuclein aggregates.

26. (canceled)

27. A method of positron emission tomography (PET) imaging of alpha-synuclein aggregates in a tissue of a subject, the method comprising the steps:
(a) Administering a compound according to claim 1 to the subject;
(b) Allowing the compound to bind to the alpha-synuclein aggregates; and
(c) Detecting the compound bound to the alpha-synuclein aggregates by collecting a positron emission tomography (PET) image of the tissue of the subject preferably wherein the tissue is a tissue of the central nervous system (CNS), an eye tissue, tissue of a peripheral organ, or a brain tissue, more preferably wherein the tissue is brain tissue.

28. (canceled)

29. A method selected from the group consisting of:
(i) A method for the detection and optionally quantification of alpha-synuclein aggregates in a tissue of a subject, the method comprising the steps:
(a) Bringing a sample or a specific body part or body area suspected to contain an alpha-synuclein aggregates into contact with a compound according to claim 1;
(b) Allowing the compound to bind to the alpha-synuclein aggregates, including but not limited to, Lewy bodies and/or Lewy neurites;
(c) Detecting the compound bound to the alpha-synuclein aggregates using positron emission tomography; and
(d) Optionally quantifying the amount of the compound bound to the alpha-synuclein aggregates;
(ii) A method of diagnosing of or determining a predisposition to a disease, disorder or abnormality associated with alpha-synuclein aggregates, the method comprising the steps:
(a) Bringing a sample or a specific body part or body area suspected to contain alpha-synuclein aggregates into contact with a compound according to claim 1;
(b) Allowing the compound to bind to the alpha-synuclein aggregates;
(c) Detecting the compound bound to the alpha-synuclein aggregates; and
(d) Optionally correlating the presence or absence of the compound bound to the alpha-synuclein aggregates with the presence or absence of the alpha-synuclein aggregates in the sample or specific body part or body area;
(iii) A method of prognosing a disease, disorder or abnormality associated with alpha-synuclein aggregates, wherein the method comprises the steps:
(a) Bringing a sample, a specific body part or body area suspected to contain alpha-synuclein aggregates into contact with a compound according to claim 1;
(b) Allowing the compound to bind to the alpha-synuclein aggregates;
(c) Detecting the compound bound to the alpha-synuclein aggregates;
(d) Optionally correlating the presence or absence of the compound bound to the alpha-synuclein aggregates with the presence or absence of the alpha-synuclein aggregates in the sample or specific body part or body area; and
(e) Optionally repeating steps (a) to (c) and, if present, optional step (d) at least one time;
(iv) A method of monitoring the progression of a disease, disorder or abnormality associated with alpha-synuclein aggregates in a patient, wherein the method comprises the steps:
(a) Bringing a sample, a specific body part or body area suspected to contain alpha-synuclein aggregates into contact with a compound according to claim 1;
(b) Allowing the compound to bind to the alpha-synuclein aggregates;
(c) Detecting the compound bound to the alpha-synuclein aggregates;
(d) Optionally correlating the presence or absence of the compound bound to the alpha-synuclein aggregates with the presence or absence of the alpha-synuclein aggregates in the sample or specific body part or body area; and
(e) Optionally repeating steps (a) to (c) and, if present, optional step (d) at least one time;
(v) A method of predicting responsiveness of a patient suffering from a disease, disorder, or abnormality associated with alpha-synuclein aggregates to a treatment of the disease, disorder, or abnormality associated with alpha-synuclein aggregates, wherein the method comprises the steps:
(a) Bringing a sample, a specific body part or body area suspected to contain alpha-synuclein aggregates into contact with a compound according to claim 1;
(b) Allowing the compound to bind to the alpha-synuclein aggregates;
(c) Detecting the compound bound to the alpha-synuclein aggregates;
(d) Optionally correlating the presence or absence of the compound bound to the alpha-synuclein aggregates with the presence or absence of the alpha-synuclein aggregates in the sample or specific body part or body area; and
(e) Optionally repeating steps (a) to (c) and, if present, optional step (d) at least one time.

30. (canceled)

31. (canceled)

32. (canceled)

33. A compound of formula (III-F) 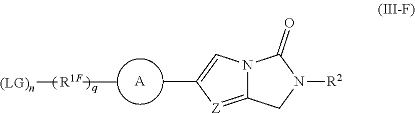
or a stereoisomer, racemic mixture, pharmaceutically acceptable salt, hydrate, or solvate thereof, wherein 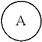
is a 6-membered heteroaryl, which is optionally substituted with at least one halo, haloC₁-C₄alkyl, haloC₁-C₄alkoxy, C₁-C₄alkoxy, or C₁-C₄alkyl;
R^{1F} is a 4- to 6-membered heterocyclyl; or
R^{1F} is C₁-C₄alkoxy, or C₁-C₄alkyl; or
R^{1F} is —NH—C₃-C₆cycloalkyl or —C₃-C₆cycloalkyl; and
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
Z is CH or N;
LG is a leaving group;
q is 0 or 1; and
n is at least 1; preferably wherein LG is selected from nitro, bromo, chloro, iodo, C₁-C₄alkylsulfonate and C₆-C₁₀arylsulfonate, wherein the C₆-C₁₀arylsulfonate can be optionally substituted with —CH₃ or —NO₂.

34. (canceled)

35. A compound of formula (I-F) 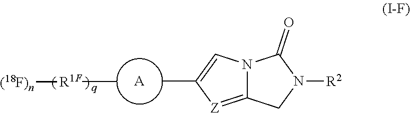
or a stereoisomer, racemic mixture, pharmaceutically acceptable salt, hydrate, or solvate thereof, wherein 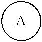
is a 6-membered heteroaryl, which is optionally substituted with at least one halo, haloC₁-C₄alkyl, haloC₁-C₄alkoxy, C₁-C₄alkoxy, or C₁-C₄alkyl;
R^{1F} is a 4- to 6-membered heterocyclyl; or
R^{1F} is C₁-C₄alkoxy, or C₁-C₄alkyl; or
R^{1F} is —NH—C₃-C₆cycloalkyl or C₃-C₆cycloalkyl;
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
Z is CH or N;
q is 0 or 1; and
n is at least 1, preferably 1.

36. A compound of formula (III-H) or (I-H) or a stereoisomer, racemic mixture, pharmaceutically acceptable salt, hydrate, or solvate thereof 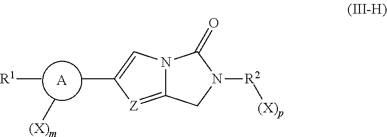
wherein 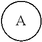
is a 6-membered heteroaryl, which is optionally substituted with at least one halo, haloC₁-C₄alkyl, haloC₁-C₄alkoxy, C₁-C₄alkoxy, or C₁-C₄alkyl;
R¹ is a 4- to 6-membered heterocyclyl which is optionally substituted with at least one halo; or
R¹ is halo, haloC₁-C₄alkyl, haloC₁-C₄alkoxy, C₁-C₄alkoxy, or C₁-C₄alkyl; or
R¹ is —NH—C₃-C₆cycloalkyl or —C₃-C₆cycloalkyl, wherein the C₃-C₆cycloalkyl of —NH—C₃-C₆cycloalkyl or —C₃-C₆cycloalkyl is optionally substituted with at least one halo;
R² is a 5-membered or 6-membered heteroaryl, optionally substituted with 1 or 2 substituents independently selected from haloC₁-C₄alkyl, haloC₁-C₄alkoxy, C₁-C₄alkoxy, and C₁-C₄alkyl;
Z is CH or N;
X is bromo, chloro or iodo;
m is 0, 1, 2 or 3;
p is 0, 1, 2 or 3; and
with the proviso that the compound of formula (III-H) comprises at least one X; 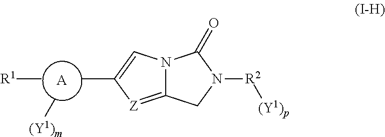
wherein 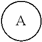
is a 6-membered heteroaryl, which is optionally substituted with at least one halo, haloC₁-C₄alkyl, haloC₁-C₄alkoxy, C₁-C₄alkoxy, or C₁-C₄alkyl;
R¹ is a 4- to 6-membered heterocyclyl which is optionally substituted with at least one halo; or
R¹ is halo, haloC₁-C₄alkyl, haloC₁-C₄alkoxy, C₁-C₄alkoxy, or C₁-C₄alkyl; or
R¹ is —NH—C₃-C₆cycloalkyl or —C₃-C₆cycloalkyl, wherein the C₃-C₆cycloalkyl of —NH—C₃-C₆cycloalkyl or —C₃-C₆cycloalkyl is optionally substituted with at least one halo;
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
Z is CH or N;
Y¹ is D, CD₃, T or CT₃;
m is 0, 1, 2 or 3;
p is 0, 1, 2 or 3; and
with the proviso that the compound of formula (I-H) comprises at least one D, CD₃, T or CT₃,
wherein D is ²D (Deuterium) and T is ³H (Tritium).

37. (canceled)

38. A method selected from the group consisting of:
(i) A method of preparing the compound of formula (I-F), the method comprising reacting the compound of formula (III-F) with a ¹⁸F-fluorinating agent, so that LG is replaced by ¹⁸F; preferably wherein the ¹⁸F-fluorinating agent is selected from K¹⁸F, Rb¹⁸F, Cs¹⁸F, Na¹⁸F, Kryptofix[222]¹⁸F, tetra(C₁₋₆alkyl)ammonium salt of ¹⁸F, and tetrabutylammonium [¹⁸F]fluoride;
(ii) A method of preparing the compound of formula (I-H), wherein Y¹ is D or CD₃, the method comprising reacting the compound of formula (III-H) with a ²H radiolabelling agent, so that X is replaced by D or CD₃; wherein the compound of formula (I-H) and the compound of formula (III-H) are as defined in claim 36; and
(iii) A method of preparing the compound of formula (I-H), wherein Y¹ is T or CT₃, comprising reacting the compound of formula (III-H) with a ³H radiolabelling agent like CT₃ radiolabelling agent, so that X is replaced by T or CT₃ wherein the compound of formula (I-H) and the compound of formula (III-H) are as defined in claim 36; 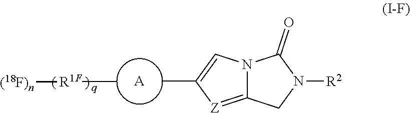
or a stereoisomer, racemic mixture, pharmaceutically acceptable salt, hydrate, or solvate thereof, wherein 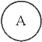
is a 6-membered heteroaryl, which is optionally substituted with at least one halo, haloC₁-C₄alkyl, haloC₁-C₄alkoxy, C₁-C₄alkoxy, or C₁-C₄alkyl;
R^{1F} is a 4- to 6-membered heterocyclyl; or
R^{1F} is C₁-C₄alkoxy, or C₁-C₄alkyl; or
R^{1F} is —NH—C₃-C₆cycloalkyl or C₃-C₆cycloalkyl;
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
Z is CH or N;
q is 0 or 1; and
n is at least 1, preferably 1; 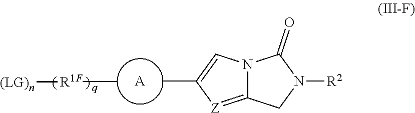
or a stereoisomer, racemic mixture, pharmaceutically acceptable salt, hydrate, or solvate thereof, wherein 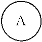
is a 6-membered heteroaryl, which is optionally substituted with at least one halo, haloC₁-C₄alkyl, haloC₁-C₄alkoxy, C₁-C₄alkoxy, or C₁-C₄alkyl;
R^{1F} is a 4- to 6-membered heterocyclyl; or
R^{1F} is C₁-C₄alkoxy, or C₁-C₄alkyl; or
R^{1F} is —NH—C₃-C₆cycloalkyl or —C₃-C₆cycloalkyl; and
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
Z is CH or N;
LG is a leaving group;
q is 0 or 1; and
n is at least 1 preferably wherein LG is selected from nitro, bromo, chloro, iodo, C₁-C₄alkylsulfonate and C₆-C₁₀arylsulfonate, wherein the C₆-C₁₀arylsulfonate can be optionally substituted with —CH₃ or —NO₂.

39. (canceled)

40. (canceled)

41. (canceled)

42. An in vitro analytical reference or an in vitro screening tool comprising the compound according to claim 1.

43. A test kit for the detection and/or diagnosis of a disease, disorder or abnormality associated with alpha-synuclein aggregates, wherein the test kit comprises at least one compound as defined in claim 9.

44. A kit for preparing a radiopharmaceutical preparation, wherein the kit comprises a sealed vial containing at least one compound selected from a compound of formula (III-F) and a compound of formula (III-H) or a stereoisomer, racemic mixture, pharmaceutically acceptable salt, hydrate, or solvate of the compound of formula (III-F) or the compound of formula (III-H): 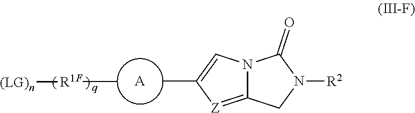
wherein 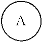
is a 6-membered heteroaryl, which is optionally substituted with at least one halo, haloC₁-C₄alkyl, haloC₁-C₄alkoxy, C₁-C₄alkoxy, or C₁-C₄alkyl;
R^{1F} is a 4- to 6-membered heterocyclyl; or
R^{1F} is C₁-C₄alkoxy, or C₁-C₄alkyl; or
R^{1F} is —NH—C₃-C₆cycloalkyl or —C₃-C₆cycloalkyl; and
R² is a 5-membered or 6-membered heteroaryl optionally substituted with 1 or 2 substituents independently selected from haloC₁-C₄alkyl, haloC₁-C₄alkoxy, C₁-C₄alkoxy, and C₁-C₄alkyl;
Z is CH or N;
LG is a leaving group;
q is 0 or 1; and
n is at least 1; preferably wherein LG is selected from nitro, bromo, chloro, iodo, C₁-C₄alkylsulfonate and C₆-C₁₀arylsulfonate, wherein the C₆-C₁₀arylsulfonate can be optionally substituted with —CH₃ or —NO₂; 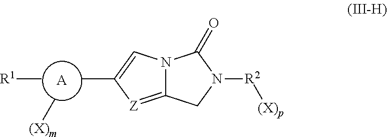
wherein 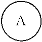
is a 6-membered heteroaryl, which is optionally substituted with at least one halo, haloC₁-C₄alkyl, haloC₁-C₄alkoxy, C₁-C₄alkoxy, or C₁-C₄alkyl;
R¹ is a 4- to 6-membered heterocyclyl which is optionally substituted with at least one halo; or
R¹ is halo, haloC₁-C₄alkyl, haloC₁-C₄alkoxy, C₁-C₄alkoxy, or C₁-C₄alkyl; or
R¹ is —NH—C₃-C₆cycloalkyl or —C₃-C₆cycloalkyl, wherein the C₃-C₆cycloalkyl of —NH—C₃-C₆cycloalkyl or —C₃-C₆cycloalkyl is optionally substituted with at least one halo;
R² is a 5-membered or 6-membered heteroaryl, optionally substituted with 1 or 2 substituents independently selected from haloC₁-C₄alkyl, haloC₁-C₄alkoxy, C₁-C₄alkoxy, and C₁-C₄alkyl;
Z is CH or N;
X is bromo, chloro or iodo;
m is 0, 1, 2 or 3;
p is 0, 1, 2 or 3; and
with the proviso that the compound of formula (III-H) comprises at least one X.

45. The kit according to claim 44, wherein:
(i) the radiopharmaceutical preparation is employed in the imaging of alpha-synuclein aggregates, wherein the imaging is preferably conducted by positron emission tomography; or
(ii) the radiopharmaceutical preparation is employed in in vitro imaging, ex vivo imaging, or in vivo imaging, preferably the radiopharmaceutical preparation is employed in in vivo imaging; or
(iii) the radiopharmaceutical preparation is employed in brain imaging.

46. (canceled)

47. (canceled)
